(19) United States
(12) Patent Application Publication
(10) Pub. No.: US 2026/0286449 A1
(43) Pub. Date: Sep. 24, 2026

(54) METHOD AND SENSOR FOR DETECTING BACTERIAL NUCLEIC ACID

(71) Applicant: NOVASCOPE BIOCHIPS INC., Dover, DE (US)

(72) Inventor: YUH-SHYONG YANG, Hsinchu City (TW)

(73) Assignee: NOVASCOPE BIOCHIPS INC., Dover, DE (US)

(21) Appl. No.: 19/476,198

(22) PCT Filed: May 8, 2024
(86) PCT No.: PCT/US24/28442

§ 371 Date: Oct. 17, 2025

Related U.S. Application Data
(60) Provisional application No. 63/500,915, filed on May 9, 2023.

(51) Int. Cl. C12Q 1/689; C12Q 1/6825

(52) U.S. Cl. CPC C12Q 1/689; C12Q 1/6825

(57) ABSTRACT

A method for detecting bacterial nucleic acid is provided. The method includes steps of providing an extended gate field-effect transistor (EGFET) having a sensing area; immobilizing a nucleic acid probe on the sensing area, wherein the nucleic acid probe is specific to Gram-negative bacteria or Gram-positive bacteria; providing a control sample on the sensing area of the EGFET; detecting a first electrical signal of the EGFET after the control sample is provided on the sensing area of the EGFET; providing a sample on the sensing area of the EGFET; detecting a second electrical signal of the EGFET after the sample is provided on the sensing area of the EGFET; and comparing the first electrical signal and the second electrical signal to determine whether there is the bacterial nucleic acid in the sample.

### CROSS-REFERENCE TO RELATED APPLICATION

This application is a U.S. national stage application under 35 U.S.C. § 371 of International Application No. PCT/US24/28442, filed May 8, 2024, which claims the benefit of U.S. Provisional Application No. 63/500,915, filed May 9, 2023, the contents of which are incorporated herein by reference.

### REFERENCE TO AN ELECTRONIC SEQUENCE LISTING

The content of the electronic sequence listing (sequencelisting.xml; size: 7.96 kb; and date of creation: Apr. 16, 2024) is herein incorporated by reference in its entirety.

### BACKGROUND OF THE INVENTION

### 1. Field of the Invention

The present disclosure relates to a method and a sensor for detecting bacterial nucleic acid, and in particular to a method and a sensor for detecting bacterial nucleic acid using EGFET.

### 2. Description of the Related Art

Sepsis is the body's extreme response to an infection, primarily due to the increase in invasive procedures, opening wounds, chemotherapy, and transplantation (Esper et al., Critical care, 2009. 13(1): p. 1-3; Minasyan et al, Scandinavian journal of trauma, resuscitation and emergency medicine, 2019. 27(1): p. 1-22). Most sepsis is caused by bacteria infections in the hospital and community, and the bacteria that cause sepsis can be divided into gram-positive bacteria (Staphylococcus aureus, Staphylococcus epidermidis, Streptococcus pneumonia, Listeria, etc.), and gram-negative bacteria (Escherichia coli, Salmonella, Klebsiella pneumonia, etc.). Among these bacteria, Escherichia coli and Staphylococcus aureus are the most common species to cause bacterial infections in sepsis (Franco-Paredes, Core concepts in clinical infectious diseases (CCCID). 2016: Academic Press; Burdino et al., Diagnostic Microbiology and Infectious Disease, 2014. 79(3): p. 287-292; Lisowska-Lysiak et al., Pol J Microbiol, 2021. 70(1): p. 13-23).

Antibiotic treatment is the most common type of drug treatment used in hospitals to fight bacterial infections. However, the drug treatment of sepsis, the delivery time, and the type of antibiotics used are closely related to the detection of sepsis and prognosis recovery. The inappropriate use of antibiotics may lead to increased adverse effects, secondary infections, and drug interactions (Llor et al., Therapeutic Advances in Drug Safety, 2014. 5(6): p. 229-241).

In clinical detection, the blood culture (gold standard) represents a reference method to detect bacteria or fungi. The sepsis detection method is still following the gold standard method of whole blood diagnosis, and other biochemical tests are used to improve the sensitivity and specificity of the detection. Although the traditional detection method (whole blood culture diagnosis) has sufficient sensitivity to detect a single bacterium, it takes extra time to grow the bacteria to amplify the signal, which is relatively time-consuming. Detection of sepsis takes a lot of time, and symptoms can worsen from days to hours for patients.

Using the 16S rRNA sequence as a way to study the detection and identification of microorganisms, due to this 16S rRNA sequence in almost all bacteria is a conserved region in bacterial genes, can be an effective way to identify bacteria (Janda et al., Journal of Clinical Microbiology, 2007. 45(9): p. 2761-2764; Srinivasan et al., PLoS One, 2015. 10(2): p. e0117617; Klausegger et al., Journal of Clinical Microbiology, 1999. 37(2): p. 464-466; Shi-qiang et al., Journal of Zhejiang University-SCIENCE A, 2000. 1(2): p. 222-226). Several research teams have previously published the use of bacterial-specific conserved region 16S rRNA sequences for clinical molecular detection of diseases and suitable targets, including clinical studies such as cystic fibrosis, endophthalmitis, and sepsis (Aarthi et al., Microbiological Research, 2013. 168(8): p. 497-503; Carroll et al., Journal of Clinical Microbiology, 2000. 38(5): p. 1753-1757; Shang et al., Pediatric Research, 2005. 58(1): p. 143-148; Abayasekara et al., BMC Infectious Diseases, 2017. 17(1): p. 631).

A design of a DNA sequence that can distinguish gram-positive and negative bacteria has been published as a probe to identify the bacterial species in the sample in real-time PCR (Burdino et al., Diagnostic Microbiology and Infectious Disease, 2014. 79(3): p. 287-292; Shang et al., Pediatric Research, 2005. 58(1): p. 143-148). The identification of gram-positive and negative bacteria can be achieved by detecting nucleic acid in bacterial samples, and this method is expected to replace the traditional Gram-stain method for identifying bacteria. The method to detect bacterial nucleic acid will reduce the time required for detection from 3-14 days to 3 days.

Field-effect transistors (FET) are one of the most powerful biosensors with excellent sensitivity and specificity and have the advantages of low-cost, real-time, and label-free biosensing applications. By modifying the surface of FET with a probe to detect targets, such as nucleic acid and proteins, the sensor can directly translate the signal of the target on the FET surface into an electronic signal (Syu et al., ECS Journal of Solid State Science and Technology, 2018. 7(7): p. Q3196-Q3207; Salami et al., Biosensors and Bioelectronics, 2019. 10: p. 1-11; Sadighbayan et al., TrAC Trends in Analytical 45 Chemistry, 2020. 133: p. 116067; Hu et al., Scientific Reports, 2018. 8(1): p. 12598).

The main applications of extended gate field-effect transistors (EGFETs) are the detection of ionic species, pH, and specifically biological molecules such as antigens, cells, nucleic acids, and proteins, through the functionalization of sensing surfaces (Pan et al., Sensors and Actuators B: Chemical, 2010. 144(1): p. 139-145; Pan et al., Journal of Alloys and Compounds, 2022. 903: p. 163955; Bartold et al., Biosensors and Bioelectronics, 2022. 208: p. 114203; Wenga et al., Biosensors and Bioelectronics, 2013. 40(1): p. 141-146; Sakata et al., Japanese Journal of Applied Physics, 2005. 44(4B): p. 2860-2863; Yin et al., Materials Chemistry and Physics, 2001. 70(1): p. 12-16).

In order to shorten the clinical detection time and rapidly distinguish the gram type of bacteria, the present disclosure provides a method and a sensor for detecting bacterial nucleic acid.

### BRIEF SUMMARY OF THE INVENTION

It is an objective of the present disclosure to provide a method for detecting bacterial nucleic acid. The method for detecting bacterial nucleic acid includes steps of: providing an extended gate field-effect transistor (EGFET) having a sensing area; immobilizing a nucleic acid probe on the sensing area, wherein the nucleic acid probe is specific to Gram-negative bacteria or Gram-positive bacteria; providing a control sample on the sensing area of the EGFET; detecting a first electrical signal of the EGFET after the control sample is provided on the sensing area of the EGFET; providing a sample on the sensing area of the EGFET; detecting a second electrical signal of the EGFET after the sample is provided on the sensing area of the EGFET; and comparing the first electrical signal and the second electrical signal to determine whether there is the bacterial nucleic acid in the sample.

In an embodiment, the nucleic acid probe includes a sequence of SEQ ID NO: 1 which is specific to the Gram-negative bacteria.

In an embodiment, the Gram-negative bacteria is E. coli.

In an embodiment, the nucleic acid probe includes a sequence of SEQ ID NO: 2 which is specific to the Gram-positive bacteria.

In an embodiment, the Gram-positive bacteria is S. aureus.

In an embodiment, the sample is diluted with Tris-HCl buffer.

In an embodiment, the Tris-HCl buffer has a concentration of 25 mM.

In an embodiment, the first electrical signal is converted to a first I_{D}-V_{G} curve, and the second electrical signal is converted to a second I_{D}-V_{G} curve.

In an embodiment, there is the bacterial nucleic acid in the sample when there is a shift from the first I_{D}-V_{G} curve to the second I_{D}-V_{G} curve.

In an embodiment, the nucleic acid probe includes a sequence of SEQ ID NO: 1 which is specific to the Gram-negative bacteria, and the bacterial nucleic acid of the Gram-negative bacteria in the sample has a concentration of at least 10 ag/μL.

In an embodiment, the nucleic acid probe includes a sequence of SEQ ID NO: 2, and the Gram-positive bacteria in the sample has a concentration of at least 1 fg/μL.

It is another objective of the present disclosure to provide a method for detecting bacterial nucleic acid. The method includes steps of: providing an extended gate field-effect transistor (EGFET) having a sensing area; immobilizing a nucleic acid probe on the sensing area, wherein the nucleic acid probe is specific to Gram-negative bacteria or Gram-positive bacteria; providing a control sample on the sensing area of the EGFET; detecting a first electrical signal of the EGFET after the control sample is provided on the sensing area of the EGFET; providing a sample on the sensing area of the EGFET; detecting a second electrical signal of the EGFET after the sample is provided on the sensing area of the EGFET; and based on the first electrical signal and the second electrical signal, determining an amount of the bacterial nucleic acid in the sample.

In an embodiment, the nucleic acid probe includes a sequence of SEQ ID NO: 1 which is specific to the Gram-negative bacteria.

In an embodiment, the Gram-negative bacteria is E. coli.

In an embodiment, the nucleic acid probe includes a sequence of SEQ ID NO: 2 which is specific to the Gram-positive bacteria.

In an embodiment, the Gram-positive bacteria is S. aureus.

In an embodiment, the sample is diluted with Tris-HCl buffer.

In an embodiment, the Tris-HCl buffer has a concentration of 25 mM.

In an embodiment, the first electrical signal is converted to a first I_{D}-V_{G} curve, and the second electrical signal is converted to a second I_{D}-V_{G} curve.

In an embodiment, the method further includes a step of comparing areas of the first I_{D}-V_{G} curve and the second I_{D}-V_{G} curve to determine the amount of the bacterial nucleic acid.

In an embodiment, the nucleic acid probe includes a sequence of SEQ ID NO: 1 which is specific to the Gram-negative bacteria, and the bacterial nucleic acid of the Gram-negative bacteria in the sample has a concentration of at least 10 ag/μL.

In an embodiment, the nucleic acid probe includes a sequence of SEQ ID NO: 2, and the Gram-positive bacteria in the sample has a concentration of at least 1 fg/μL.

It is another objective of the present disclosure to provide a sensor for detecting bacterial nucleic acid. The sensor includes an extended gate field-effect transistor (EGFET) having a sensing area; and a nucleic acid probe immobilized on the sensing area, wherein the nucleic acid probe is specific to Gram-negative bacteria or Gram-positive bacteria.

In an embodiment, the nucleic acid probe includes a sequence of SEQ ID NO: 1 which is specific to the Gram-negative bacteria.

In an embodiment, the Gram-negative bacteria is E. coli.

In an embodiment, the nucleic acid probe includes a sequence of SEQ ID NO: 2 which is specific to the Gram-positive bacteria.

In an embodiment, the Gram-positive bacteria is S. aureus.

In an embodiment, the bacterial nucleic acid is detected by steps of providing a control sample on the sensing area of the EGFET; detecting a first electrical signal of the EGFET after the control sample is provided on the sensing area of the EGFET; providing a sample on the sensing area of the EGFET; and detecting a second electrical signal of the EGFET after the sample is provided on the sensing area of the EGFET.

In an embodiment, the first electrical signal is converted to a first I_{D}-V_{G} curve, and the second electrical signal is converted to a second I_{D}-V_{G} curve.

In an embodiment, there is the bacterial nucleic acid in the sample when there is a shift from the first I_{D}-V_{G} curve to the second I_{D}-V_{G} curve.

In an embodiment, areas of the first I_{D}-V_{G} curve and the second I_{D}-V_{G} curve are compared to determine an amount of the bacterial nucleic acid.

It is another objective of the present disclosure to provide a method for verifying an electrical property of an EGFET. The method includes steps of: providing an EGFET in a first buffer solution with a first pH value; measuring a first electric signal of the EGFET in the first buffer solution; providing a second buffer solution with different pH values; providing the EGFET in the second buffer solution; measuring a second electrical signal of the EGFET in the second buffer solution; and converting the first electric signal to a first I_{D}-V_{G} curve and converting the second electrical signal to a second I_{D}-V_{G} curve.

In an embodiment, one of the first buffer solution, the second buffer solution and the third buffer solution is BTP buffer.

In an embodiment, the BTP buffer has a concentration of 10 mM.

In an embodiment, the different pH values include 5, 7 and 8.

In an embodiment, the EGFET is measured in the buffer solution with the different pH values in the order of pH7, pH5, pH7, pH8, and pH7.

In an embodiment, the EGFET is a nude EGEFT.

It is another objective of the present disclosure to provide a method for verifying surface modification of an EGFET. The method includes steps of: providing an EGFET in a buffer solution with a pH value; measuring a first electric signal of the EGFET in the buffer solution with the pH value; modifying the EGFET to form a modified EGFET; providing the modified EGFET in the buffer solution with the pH value; measuring a second electrical signal of the modified EGFET in the buffer solution with the pH value; converting the first electric signal to a first I_{D}-V_{G} curve; and converting the second electrical signal to a second I_{D}-V_{G} curve.

In an embodiment, the buffer solution is BTP buffer.

In an embodiment, the BTP buffer has a concentration of 10 mM.

In an embodiment, the EGFET is a nude EGEFT.

### BRIEF DESCRIPTION OF THE DRAWINGS

FIG. 1A and FIG. 1B are images illustrating an extended gate field effect transistor (EGFET); and FIG. 1C is a schematic view illustrating that the EGFET is composed of MOSFET and an extended sensing membrane.

FIG. 2 is a schematic view illustrating a sensing system for DNA detection according to the present disclosure.

FIG. 3 is a schematic view illustrating chemical reactions according to the present disclosure.

FIG. 4 is a schematic view illustrating the processing before SEM analysis for verification of the immobilized probes according to the present disclosure.

FIG. 5A to FIG. 5C show the confirmation of different sizes of DNA by electrophoresis, in which FIG. 5A shows 55 base pairs synthetic DNA: gram-negative target DNA (lane 1) and gram-positive target DNA (lane 2); FIG. 5B shows gDNA extracted from E. coli (lane 1) and gDNA extracted from S. aureus (lane 2); and FIG. 5C shows after heat lysis, the gDNA broke into fragment DNA, and the size of DNA is shorter than 4000 base pairs, gDNA extracted from E. coli (lane 1) and gDNA extracted from S. aureus (lane 2).

FIG. 6A and FIG. 6B show the confirmation of the primers by PCR, which indicates using the GN primers can amplify the E. coli with the size of DNA being 252 base pairs (FIG. 6A), and using the GP primers can amplify the S. aureus with the size of DNA being 900 base pairs (FIG. 6B).

FIG. 7A to FIG. 7D are diagrams showing confirmation of the electrical properties of nude EGFET by measuring electrical signals, in which FIG. 7A and FIG. 7B show good electrical property of EGFET, i.e. a stable baseline under normal conditions, and FIG. 7C and FIG. 7D show a poor electrical property of EGFET, i.e. an unstable baseline under abnormal conditions (reaction condition: 10 mM BTP buffer, Vd: 2V, VG: 0-3V).

FIG. 8A and FIG. 8B are diagrams showing confirmation of the electrical properties of the EGFET according to the present disclosure.

FIG. 9A to FIG. 9D are diagrams showing verification of surface modification by step-by-step electrical measurement according to the present disclosure.

FIG. 10 A to FIG. 10F show verification of surface modification by SEM according to the present disclosure.

FIG. 11A and FIG. 11B are diagrams showing the specificity of EGFET to 55 base pairs of target DNA according to the present disclosure.

FIG. 12A and FIG. 12B are diagrams showing the specificity of EGFET to gDNA according to the present disclosure.

FIG. 13A to FIG. 13F are diagrams showing the sensitivity of EGFET to 55 base pairs of target DNA according to the present disclosure.

FIG. 14A to FIG. 14F are diagrams showing the sensitivity of EGFET to gDNA of bacteria according to the present disclosure.

FIG. 15A to FIG. 15D are diagrams showing which buffer solution can cause the least non-specific binding according to the present disclosure.

FIG. 16A and FIG. 16B are diagrams showing the specificity of EGFET to gDNA of bacteria spike-in 100× diluted finger-prick blood according to the present disclosure.

FIG. 17A to FIG. 17F are diagrams showing the sensitivity of EGFET to gDNA of bacteria spike-in 100× diluted finger-prick blood according to the present disclosure.

FIG. 18A to FIG. 18F are diagrams showing the sensitivity of EGFET to bacteria samples spike-in 100× diluted finger-prick blood according to the present disclosure.

FIG. 19A to FIG. 19C show confirmation of the target DNA by SEM according to the present disclosure.

### DETAILED DESCRIPTION OF THE INVENTION

To facilitate understanding of the objects, characteristics and effects of the present disclosure, embodiments together with the attached drawings for the detailed description of the present disclosure are provided.

### Materials

3-Aminopropyltriethoxysilane (APTES) solution, glutaraldehyde (GA) solution, sodium cyanoborohydride (NaBH3CN), bis-tris propane (BTP), streptavidin-conjugated nanogold (~20 nm colloidal gold) were purchased from Sigma-Aldrich (USA). Tris powder was purchased from J. T. Baker (USA). Acetone and ethanol (98% & 99.5% & 99.8%) were purchased from Echo Chemical Company. All solutions were diluted with DI water (resistance of water was 18.2 Q) from an ultra-pure water system (Millipore). Pure oxygen and nitrogen were purchased from the Xin Fu Fa Company. Silicone elastomer Sylgard 184 A & B was purchased from SIL-MORE. Probe DNA, target DNA, and primer sequences were based on the design of the previous studies (Aarthi et al., Microbiological Research, 2013. 168(8): p. 497-503) and Primer-Blast (Ye et al., BMC Bioinformatics, 2012. 13(1): p. 134). All synthetic oligonucleotides were synthesized by Bio-Protech Technology Enterprise. DH5α Escherichia coli (E. coli), Staphylococcus aureus (S. aureus), and fingertip blood were from the American Type Culture Collection or provided by Chang Gung Memorial Hospital and both were cultured in the lab. DNA Mini Kit was purchased from Qiagen. 100 bp DNA Ladder H3 RTU (Ready-to-Use) was purchased from GeneDirex. Healthview Nucleic Acid Stain was purchased from P & C Biotech. One Taq Quick-Load 2× Master Mix with Standard Buffer was purchased from Biolabs.

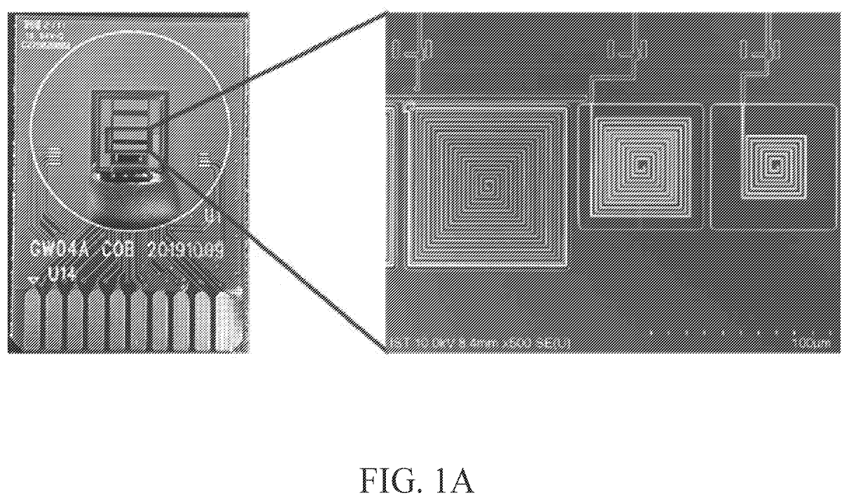

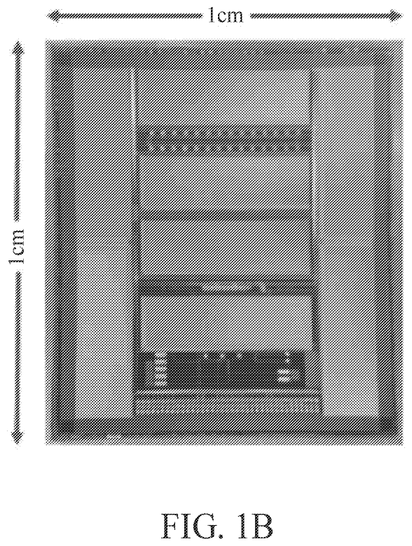

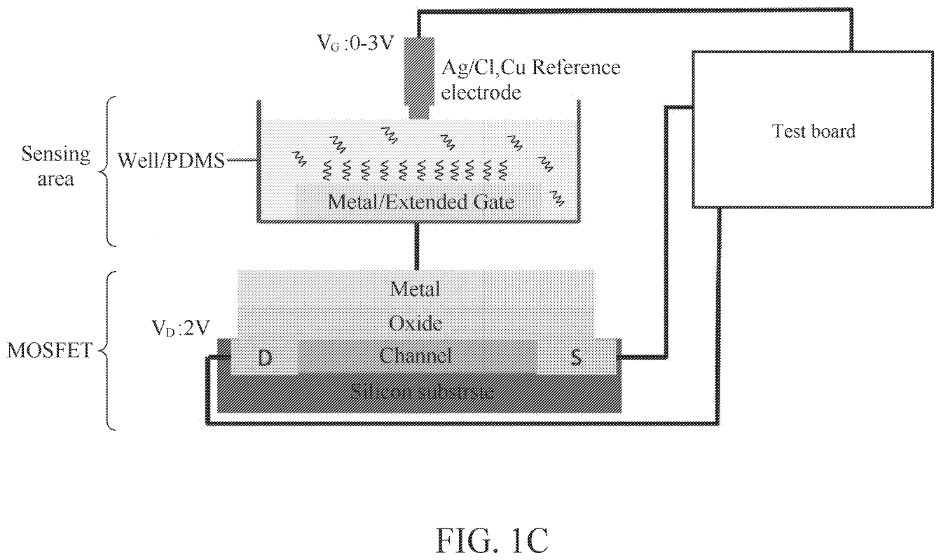

The EGFET was provided by SUNPLUS TECHNOLOGY (Hsinchu, Taiwan), and the wafer was fabricated using standardized semiconductor process technology. FIG. 1A and FIG. 1B are images illustrating an extended gate field effect transistor (EGFET), wherein the box, indicating the position of the sensing areas, on the left side of FIG. 1A is enlarged under the SEM shown at the right side of FIG. 1A to show the component structure. The length and width of the dies are 1 cm×1 cm, and the sensing device are 100 μm×100 μm as shown in FIG. 1A and FIG. 1B. As shown in FIG. 1C, the EGFET is composed of MOSFET and an extended sensing membrane, the detection principles of EGFET and ISFET are similar in that they both adjust the gate potential through the electrochemical potential change between the electrolyte and the sensing membrane. Therefore, it changes the channel conductance between the source and drain of the MOSFET and affects the drain current. The I_{D}-V_{G} signal is affected by the charges collected by the floating gate and through affecting the conductance in the channel, is passed to the test board, resulting in a shift in the electrical curve (Guliga et al., Extended gate field effect transistor (EGFET) integrated readout interfacing circuit for pH sensing. in 2014 2nd International Conference on Electrical, Electronics and System Engineering (ICEESE). 2014; Dorfman et al., Biomicrofluidics, 2020. 14(1): p. 011301).

### Open-Well System

The open-well system is made up of polydimethylsiloxane (PDMS) as a loading samples channel, which is made by mixing Sylgard 184 A and Sylgard 184 B with a ratio of 10:1. A vacuum pump is used to remove air bubbles from the gel-like mixture. The mixture is poured into a mold that forms an open-well and then heated for 10 minutes at 120° C. and placed at room temperature overnight for solidification. The PDMS is solid and cut into shape to be ready for use. The PDMS is attached to the top of the sensing area of the chip and the PDMS channel can be loaded with a 100 L sample and operated on the probe station to measure the electrical signal.

### Sensing System

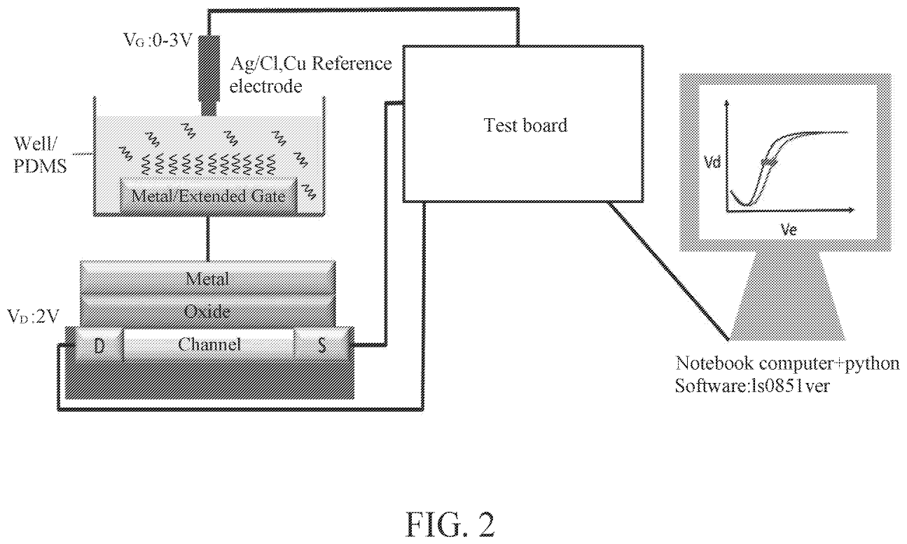

The sensing system is shown in FIG. 2. The sensing system contains a testing board that measures the electrical characteristics of the chip, a computer with the Python software Is0851ver that analyze the electrical signals, and an open-well system. The probe station is set up in a sealable metal chamber that provides an electrostatic shield to reduce outside noise. The sensor has a constant drain voltage (V_{D}) of 2V and a gate voltage (V_{G}) sweep of 0 V to 3 V is applied to the EGFET sensor, the testing board measures the I_{D}-V_{G} response. The chip designed by Sunplus (Hsinchu, Taiwan) is an integrated circuit signal, not a pure FET device. The electrical signal is measured as I_{D}-V_{G} curve and converted into the Vₒᵤₜ-V_{G} curve through the formula (1-1). The difference between the area of baseline with each concentration is calculated. The significant difference is defined as the area difference of the target sample being at least three times the area difference of the negative control sample.

$${V}_{out} = {I}_{D} \times R , \qquad ( formula 1 - 1 )$$

in which R is the electrical resistance value.

### Electrical Measurement of EGFET

Before testing the electrical signal measurement, 10 minutes are given to settle the system. After that, three successive overlapping I_{D}-V_{G} curves are obtained. The baseline of the I_{D}-V_{G} curve is stable by measuring under the 10 mM BTP, pH 7 buffer environment. Then, the 10 mM BTP buffer is removed from the open-well. The negative control sample is added to the sample loading well and incubated at room temperature for 30 minutes. After 30 minutes, the negative control sample is removed and the well is washed with 10 mM BTP buffer with pipetting three times to remove any non-specificity binding. After 10 minutes for the system to settle, the I_{D}-V_{G} response is measured. The determination of the I_{D}-V_{G} curve measurement was obtained from three successive overlapping I_{D}-V_{G} curves to make sure there is no signal variant. Next, the negative control sample is replaced with bio-samples, and following the negative control, sample steps are repeated, starting with the low concentration to the high concentration.

### Surface Modification of EGFET

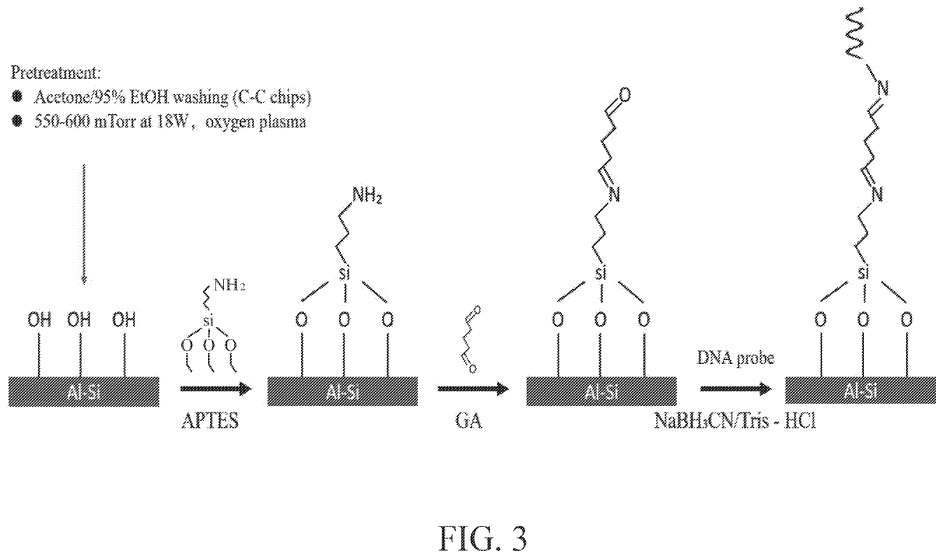

The surface immobilization process utilizes self-assembled monolayers (SAMs) technology to attach and immobilize the nucleic acid probes on the surface of the EGFETs (Hasan et al., 6-Self-assembled monolayers in biomaterials, in Nanobiomaterials, R. Narayan, Editor. 2018, Woodhead Publishing. p. 137-178.; Murugan et al., Int J Biomed Eng Technol, 2009. 2(2): p. 104-134). FIG. 3 shows illustration of chemical reactions.

Chip cleaning is performed as follows. The chip is immersed in acetone with sonication for 10 minutes, rinsed with 95% ethanol, and immersed in 95% ethanol with sonication for 10 minutes. These organic solvents are used to remove contaminating particles on the surface of the chip, and then the chip is heated at 90° C. for 1 minute to remove the surplus ethanol.

The oxygen plasma treatment is performed as follows. After the solution cleaning steps, the chip is placed into the plasma chamber of Plasma cleaner PDC-32G (HARRICK PLASMA). The excess air is sucked out, causing a vacuum that seals the chamber, and pure oxygen is pumped inside. After that, the oxygen amount was to 500-600 mTorr, and a high-level magnetic field was turned on for 1 minute. Oxygen plasma treatment not only cleans the chips but also causes the surface of the chip to become more hydrophilic by forming hydrophilic groups (—OH group) on the surface of the chip. This step can improve absorption for subsequent coating or the bond of functional groups.

APTES modification is performed as follows. The chip is immersed in an APTES solution (2% in 99.8% absolute ethanol) at room temperature with 60 rpm shaking for 30 minutes to form an amino-terminal monolayer on the chip surface. The chip is then cleaned with 99.5% ethanol three times and sonicated in 99.5% ethanol for 10 minutes to remove the remaining APTES residue. Finally, the chip is baked-dried at 120° C. for 10 minutes to eliminate surplus ethanol to induce the formation of amine groups to attach one end of the APTES on the surface.

Glutaraldehyde modification is performed as follows. The chip with an amino-terminated is immersed in 2.5% GA solution in 10 mM BTP buffer at room temperature with 60 rpm shaking for 1 hour (in the dark) to form a terminal aldehyde group. The chip is rinsed three times with 10 mM BTP buffer and blown dry with nitrogen gas.

DNA probe immobilization is performed as follows. The chip was incubated with a 100 nM amine-modified (5′-C6-NH) DNA probe in 10 mM BTP buffer at 4° C. overnight. The sequences of DNA probes are shown in Table 1, in which probe 1 is 5′-aminomodified gram-negative DNA probe (length: 37 bases), probe 2 is 5′-aminomodified gram-positive DNA probe (length: 37 bases), target 1 is gram-negative target DNA (length: 55 bases), and target 2 is gram-positive target DNA (length: 55 bases). The amines in the DNA probe will attach to the terminal-aldehyde group leftover by GA.

TABLE 1

| Number | |
|---|---|
| Probe 1 | ACCGCGAAAAGCACCGGCTACGTAGGCGGTTAAGTCG |
| (SEQ | |
| ID | |
| NO: 1) | |
| Probe 2 | AAGGGGATAACTGGGAAACGGGCTAATACCGATAGAT |
| (SEQ | |
| ID | |
| NO: 2) | |
| Target 1 | TTGACCCGGGATTTCACACGACTTAACCGCCTACGTA |
| (SEQ | GCCGGTGCTTTTCGCGGT |
| ID | |
| NO: 3) | |
| Target 2 | CCTTTCTTGACATGCCAAATCTATCGGTATTAGCCCG |
| (SEQ | TTTCCCAGTTATCCCCTT |
| ID | |
| NO: 4) | |

After 12 to 16 hours, the chip is rinsed with 10 mM BTP buffer and 10 mM Tris buffer three times. The chip is immersed in 4 mM NaBH₃CN prepared with 10 mM Tris buffer for 30 minutes to stabilize the DNA probes and block the untreated aldehyde groups. Then, the chip is rinsed with 10 mM BTP buffer three times and blown dry with nitrogen gas.

### Verification of Surface Modification

To confirm the immobilization process used in surface modification is sufficient to attach the probes to the surface of the EGFETs, two verification methods are designed. The first verification method uses the electrical signals of the chip to confirm the electric characteristic changes during each step of surface modification and the second verification method uses scanning electron microscopy (SEM).

Verification by step-by-step measurement: the chip surface modification is confirmed by measuring the I_{D}-V_{G} curve of the same chip after each surface immobilization step. Measuring the electrical properties after different stages in the modification and sensing process, chip cleaning, APTES, GA, and the immobilization of probes. After acetone and ethanol cleaning, the chip is dried with nitrogen gas, then 10 mM, pH 7 BTP buffer is loaded to obtain three overlapping I_{D}-V_{G} curves. The next step in surface modification is performed with APTES, and the mixture is heated at 120° C. for 10 minutes and then added with 10 mM, pH 7 BTP buffer, so as to obtain three overlapping I_{D}-V_{G} curves. After that, the surface modification in GA solution is completed and then washed with 10 mM, pH7 BTP buffer three times, and the chip is blown dry with nitrogen gas. A drop of 10 mM, pH 7 BTP buffer is loaded, and three overlapping I_{D}-V_{G} curves are obtained. In the final step, the probe is immobilized on the surface of the chip overnight. After overnight, the chip is rinsed with 10 mM, pH 7 BTP buffer and blown dry with nitrogen gas, and then 10 mM, pH 7 BTP buffer is loaded to obtain three overlapping I_{D}-V_{G} curves.

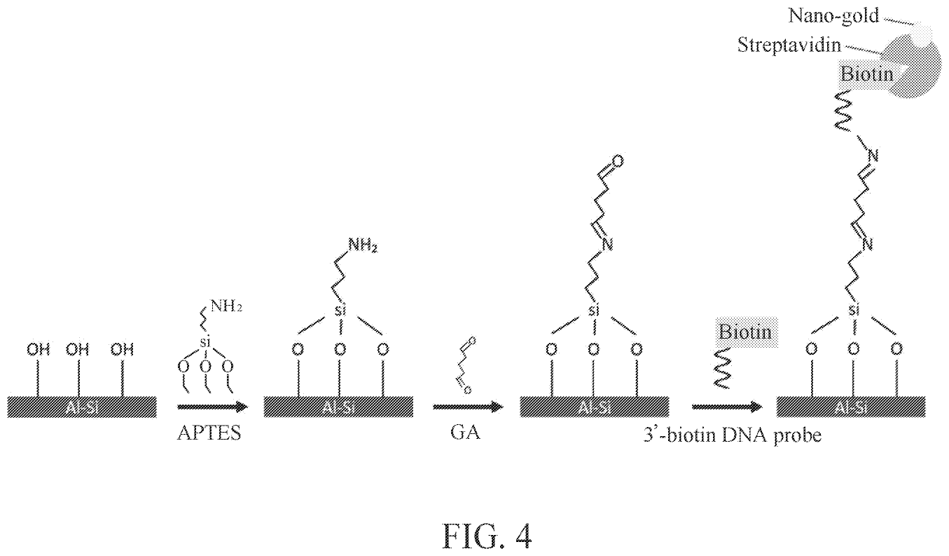

Verification by scanning electrical microscope (SEM): each step in the surface modification of EGFET for SEM analysis is needed and that is as described in the following steps: (1) chip cleaning, (2) APTES modification, (3) glutaraldehyde modification, and (4) DNA captured probe immobilization, all steps are incubated in streptavidin-conjugated nanogold solution (the 1/10 dilution with nanogold dilution buffer) at room temperature for 3 hours. After incubating, the chip is washed with nanogold washing buffer and DI water to clean the non-specific binding. For the SEM analysis, the chip is sputter-coated with platinum (Pt) before SEM scanning. The SEM is conducted by Integrated Service Technology (Hsinchu, Taiwan). Gold nanoparticles are observed under SEM images to verify the presence of DNA probes. The DNA probes listed in Table 1 are modified with the biotin group on the 3′ terminal to form 3′-biotinylated gram-negative DNA probe, 3′-biotinylated gram-positive DNA probe, 3′-biotinylated gram-negative target DNA and 3′-biotinylated gram-positive target DNA, respectively, which can catch the streptavidin-conjugated nanogold (FIG. 4).

### Sample Preparation

Bacteria preparation and quantitation: the bacteria quantitation by cell culture is done before each biosensing. The E. coli and S. aureus are suspended in 10 mM BTP buffer and then tested for their OD₆₀₀ unit using a UV-vis spectrophotometer (UV-3300, Hitachi). For E. coli and S. aureus, an OD₆₀₀ unit of 1 corresponds to the cell density of 8×10⁸ CFU/mL and 7×10⁸ CFU/mL, respectively. After that, the samples are diluted to various cell densities with 10 mM BTP buffer for testing and extraction.

Genomic DNA (gDNA) extraction: the gDNA is extracted from E. coli and S. aureus, both of which are major for causing sepsis. Quantitative cell density is 8×10⁸ CFU/mL and follows the manufacturer's instruction of DNA Mini Kit (Qiagen) steps to extract bacterial genomic DNA. The quality and purity of the genomic DNA are determined by reading the absorbance at 260 nm and the ratio of absorbance at 260 nm and 280 nm using Nanodrop, respectively. The extracted gDNA is stored at −20° C. for further usage.

### PCR Used for Confirmation of the Primers

The reaction mixture for the PCR of the 16S rRNA region of bacteria consists of a 15 μL reaction with 5.9 μLDI water, 7.5 μL One Taq Quick-Load 2× Master Mix with Standard Buffer, 0.3 μL (10 mol) of each primer (Table 2), and 1 L DNA with the thermal profile of initial denaturation at 94° C. for 5 min followed by 30 cycles of denaturation at 94° C. for 30 seconds, annealing at 61° C. for 30 seconds with gram-negative primer (the annealing temperature of the gram-positive primer is 52° C.), extension at 68° C. for 1 minute, and final extension at 68° C. for 5 minutes. The PCR products are separated by DNA gel electrophoresis with a 1.5% agarose gel, in 1×TAE buffer under 110V for 25 minutes with a PowerPac Basic Power Supply (Bio-Rad).

TABLE 2

| Number | |
|---|---|
| SEQ ID NO: 5 | ACCGCGAAAAGCACCGGCTA |
| SEQ ID NO: 6 | AGGGGTGCGAAAGCGCGGAT |
| SEQ ID NO: 7 | AAGGGGATAACTGGGAAACG |
| SEQ ID NO: 8 | AGGCTTTGGAAGTAGGAGTG |

In Table 2, SEQ ID NO: 5 indicates Gram-negative forward primer, SEQ ID NO: 6 indicates Gram-negative reverse primer, SEQ ID NO: 7 indicates Gram-positive forward primer, and SEQ ID NO: 8 indicates Gram-positive reverse primer.

Sample Processing and Detection Procedures for gDNA Extraction in Finger-Prick Blood

In the present disclosure, two methods for sample processing and detection are provided for detecting and differentiating Escherichia coli and Staphylococcus aureus in blood. One of the methods is to directly detect bacterial nucleic acids in the blood, and the other method is to pre-process the blood-containing bacteria sample for detection.

Spike-in extraction gDNA in finger-prick blood: because the blood of patients with sepsis contains free DNA, the bacteria causing sepsis can be identified by detecting bacterial DNA in the blood (Gutierrez et al., Open Forum Infectious Diseases, 2019. 6(4); Opota et al., Clinical Microbiology and Infection, 2015. 21(4): p. 323-331). The extracted bacterial genomic DNA is diluted to several concentrations and heated at 95° C. for 10 minutes and cold shocked for 3 minutes to unwind DNA double strands. The gDNA was added into the 100× diluted finger-prick blood with 25 mM Tris-HCl buffer. After the baseline of the I_{D}-V_{G} curve is stable, a drop of 100× diluted finger-prick blood is loaded into the well and incubated at room temperature for 15 minutes. After 15 minutes, the 100× diluted finger-prick blood is removed and the well is washed with 10 mM BTP buffer and 10 mM BTP+0.05% Tween-20 with a pipetting three times to remove any non-specificity binding before measuring the I_{D}-V_{G} curve. The first sample is the negative control. A certain concentration of negative control is loaded into the open well for 30 minutes; after 30 minutes, the negative control sample is removed and the well is washed with BTP buffer. After 10 minutes to settle the system, the I_{D}-V_{G} curve is measured. Next, the negative control sample is replaced with bio-samples, and following the negative control, sample steps are repeated, starting with the low concentration to the high concentration.

Spike-in bacteria in finger-prick blood: after the bacteria quantitation using the UV-vis spectrophotometer, all samples are diluted to various cell densities in the 100× diluted whole blood with 25 mM Tris-HCl buffer. After that, these samples are heated lysis at 95° C. for 10 minutes and cold shocked for 3 minutes to break down the membrane of the cell. The samples are put in the centrifuge for 14,000 rpm, 10 minutes, and aspirated with the supernatant (including the gDNA of the bacteria) for detection (Pilecky et al., Diagnostic Microbiology and Infectious Disease, 2019. 94(1): p. 7-14; Millar et al., Journal of Microbiological Methods, 2000. 42(2): p. 139-147; Dalla-Costa et al., Journal of Microbiological Methods, 2017. 140: p. 61-6). The electrical measuring steps follow the spike-in extraction gDNA steps in finger-prick blood.

### Confirmation of the Length of DNA

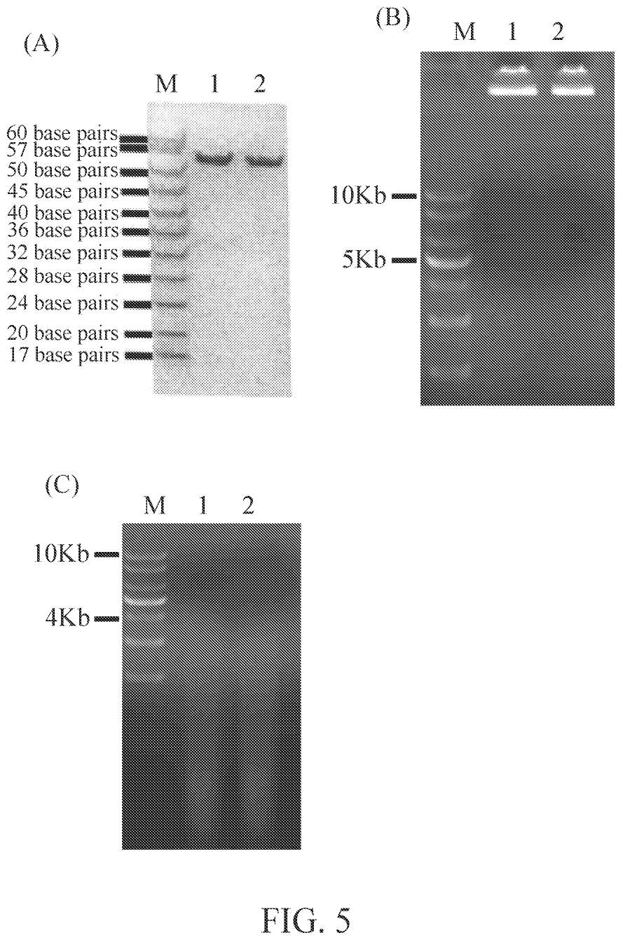

The size of DNA length is determined by gel electrophoresis. The band showed that both gram-positive and gram-negative bacteria synthetic DNA have 55 base pairs in FIG. 5A. The extraction of gDNA from E. coli and S. aureus are shown in FIG. 5B. The gDNA is heated at 95° C. for 10 minutes and cold shocked for 3 minutes to unwind DNA double strands before biosensing, and the size of the gDNA is broken into DNA fragments shown in FIG. 5C.

Confirmation of the Primers by PCR with Gel Electrophoresis

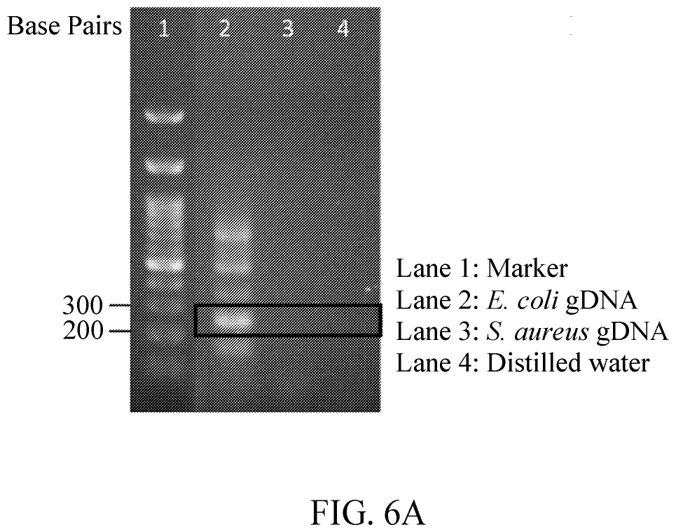

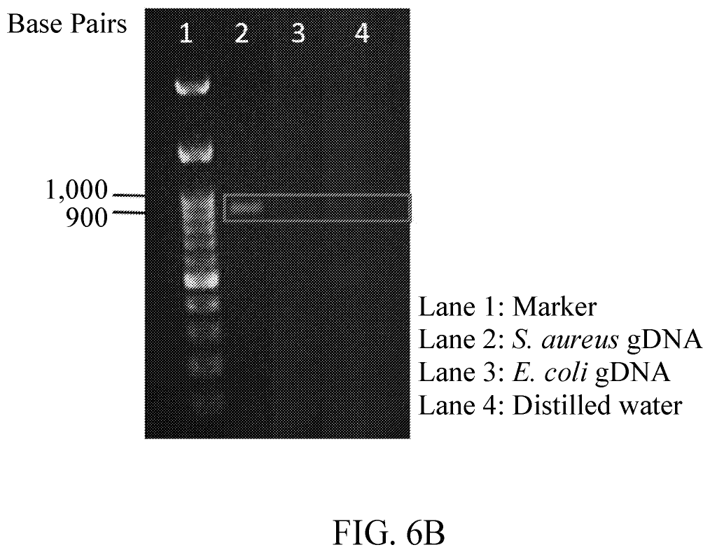

The designed primers effectively distinguish gram-positive bacteria and gram-negative bacteria as shown in FIG. 6A and FIG. 6B. In FIG. 6A, using the GN-primers can amplify the 16S rRNA sequences of gram-negative bacteria, wherein the band size is about 252 base pairs, and can't amplify the sequences of positive bacterial sequences. In FIG. 6B, using the GP-primers can amplify the 16S rRNA sequences of gram-positive bacteria, wherein the size of the band is about 900 base pairs, and can't amplify the sequences of negative bacteria. These results show that both sets of primers can distinguish the gram properties of bacteria, and the properties of these two primers can be used to design probe sequences to be immobilized on the chip.

### The Electrical Characteristics of EGFET in Aqueous Solution

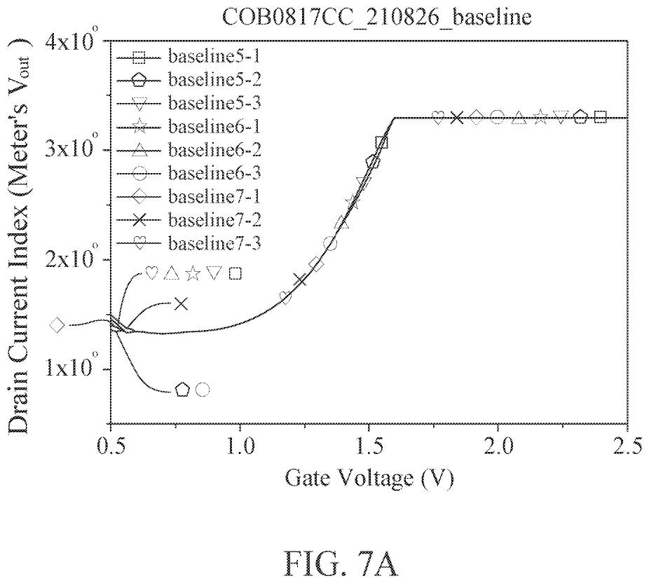

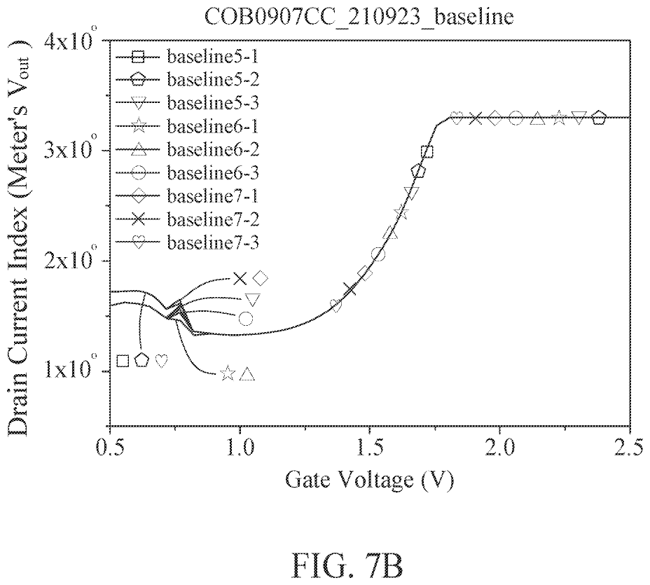

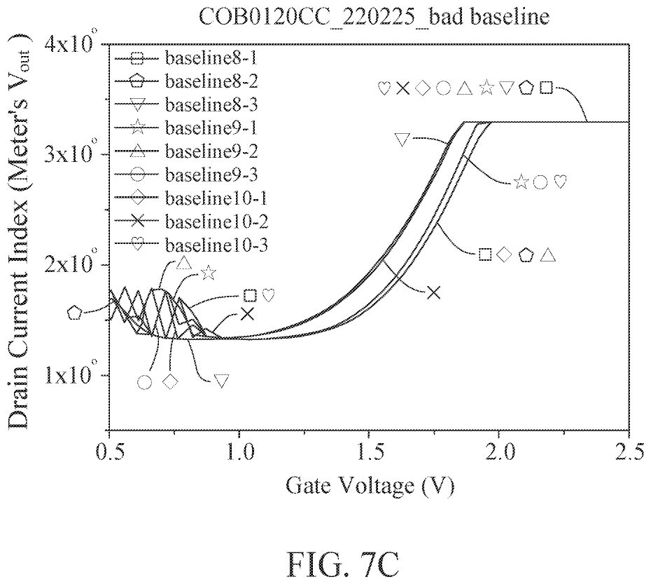

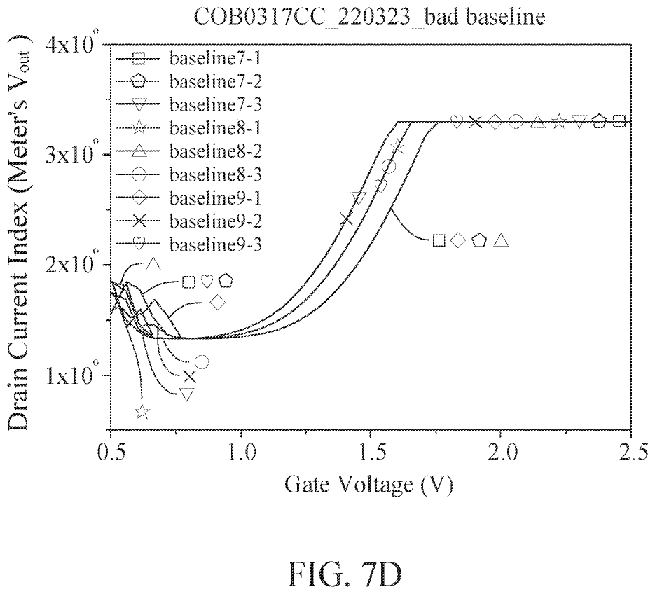

Nude Chip of EGFET Testing in Solution Environment: to confirm whether the device of the EGFET has a good electrical response after the fabrication and packaging process, the I_{D}-V_{G} curves of the nude device are measured. The electrical properties of the nude devices are analyzed in an aqueous environment (10 mM BTP, pH7). The I_{D}-V_{G} curves are measured by the constant drain voltage (V_{d}) of 2 V and a gate voltage (V_{G}) sweep of 0 V to 3 V from the EGFET sensor. The potential from the gate electrode can control the current of the devices. The I_{D}-V_{G} curves are obtained by measuring in 10 mM BTP buffer and incubated for 10 minutes before each test. The electrical signal curve of the baseline that is stable under normal conditions is shown in FIG. 7A and FIG. 7B, while the unstable under abnormal conditions is shown in FIG. 7C and FIG. 7D.

Under normal conditions, a stable baseline can be measured in six or seven rounds, whereas a baseline under abnormal conditions requires at least ten rounds to measure stability. The unstable shift of electrical curve may be caused by the production status of the wafer or caused by the improper operation, the displacement and water leakage of the mold PDMS during the measurement.

For this reason, we found that measuring the electrical properties of the nude chip before the immobilization process, which can cause the electrical measurement after the immobilization, would have a faster and more stable baseline.

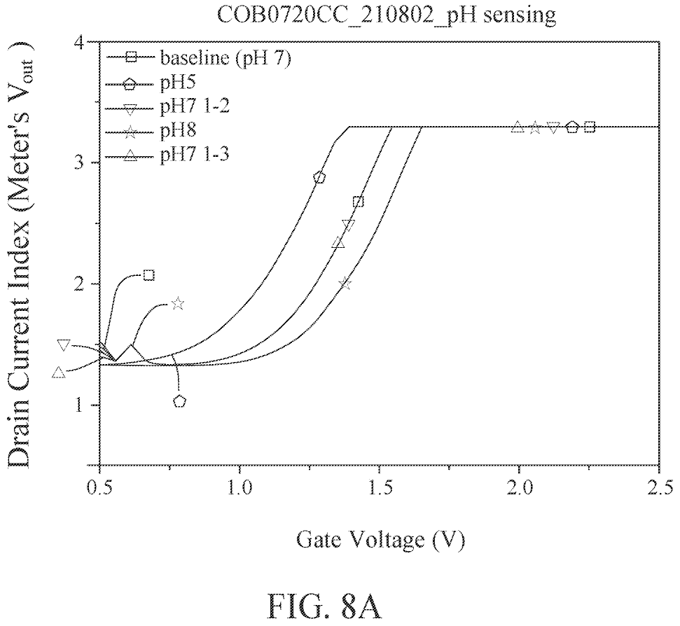

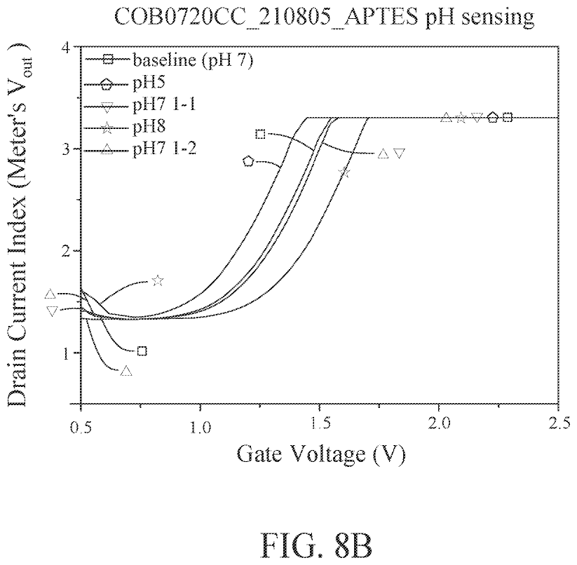

Nude and APTES-Modified EGFET pH Sensing: the electrical properties need to be measured under different pH solutions to ensure the sensor can detect the environmental ions change. The nude of EGFET is measured in the 10 mM BTP buffer with different pH values. As shown in FIG. 8A, the I_{D}-V_{G} curves shifts to the left when the buffer is changed to the 10 mM, pH 5 BTP buffer, and I_{D}-V_{G} curves shifts to the right when the buffer is changed to the 10 mM, pH 8 BTP buffer. The pH value of buffer for detection is changed in the order of pH7, pH5, pH7, pH8, and pH7.

The APTES-modified EGFET is measured in 10 mM BTP buffer with different pH values. As shown in FIG. 8B, the I_{D}-V_{G} curves shifts to the left when the buffer is changed to the 10 mM, pH 5 BTP buffer, and I_{D}-V_{G} curves shifts to the right when the buffer is changed to the 10 mM, pH 8 BTP buffer. The pH value of buffer for detection is changed in the order of pH7, pH5, pH7, pH8, and pH7. Because APTES has an amine group on the terminal that is easily protonated to produce positive charges in acidic solution, it increases drain current resulting in the I_{D}-V_{G} curves shifting to the left. These results show that the EGFET is sensitive to the potential change on the surface.

### Confirmation of Surface Modification

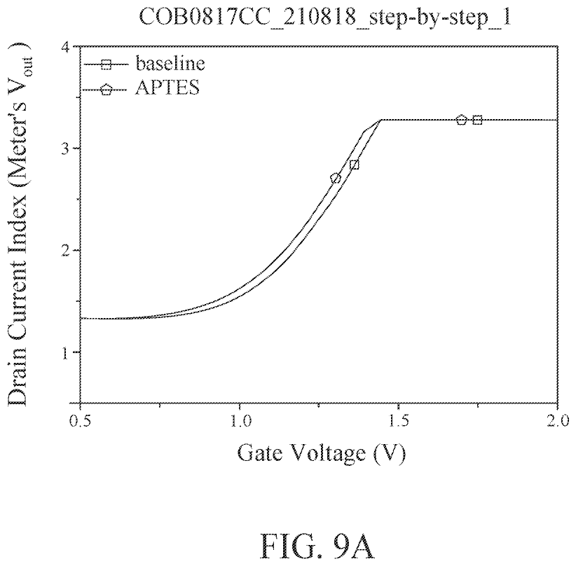

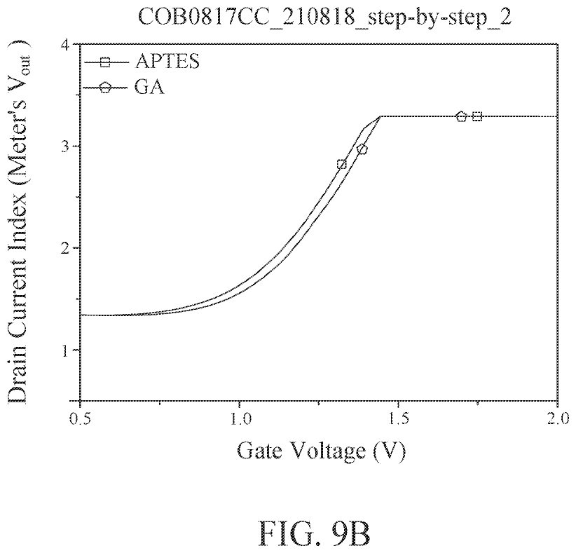

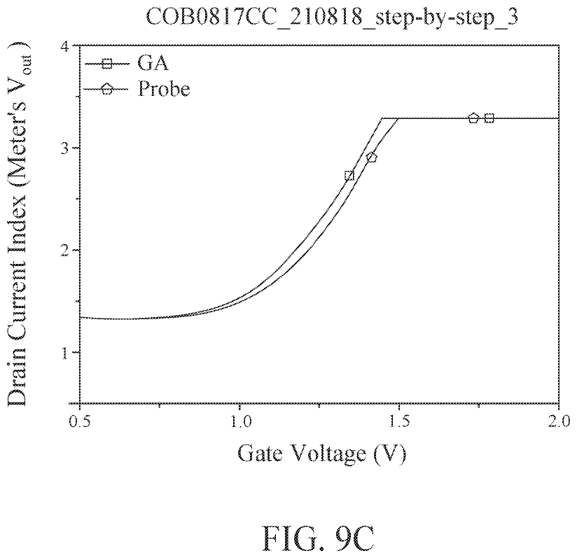

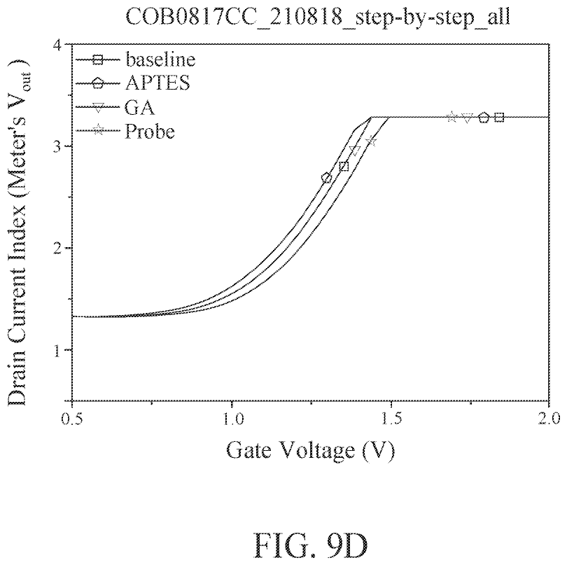

Step-by-Step Electrical Measurement: the surface modification affects the potential of the EGFET. After all steps of surface modification, the electrical response of the I_{D}-V_{G} curves is measured. First, a baseline is established after the step of chip cleaning (FIG. 9A). After APTES-modification, the I_{D}-V_{G} curve shifted to the left, because of the positive charge of the amine group of the APTES terminal (FIG. 9A). After GA-modification, the I_{D}-V_{G} curve shifted to the right, because of the un-charged glutaraldehyde bound onto the positive charge of APTES (FIG. 9B). Finally, after the immobilization of the DNA probe, the phosphate backbone of DNA carries negative charges causing the I_{D}-V_{G} curve to shift to the right (FIG. 9C). Based on the shift of the I_{D}-V_{G} curve, the immobilization of the DNA probe on the surface of the EGFET is confirmed.

The influence of each step of the reaction on the chip during the immobilization process is measured and presented by the electrical properties of I_{D}-V_{G} curve shown in FIG. 9D. The amino group of the APTES terminal has a positive charge, the aldehyde group of the GA terminal is un-charged, and the DNA probe has a negative charge. These reactants are charged and cause the I_{D}-V_{G} curves to shift. The immobilization process and the efficiency of DNA probe immobilization are verified by measuring the electrical curve.

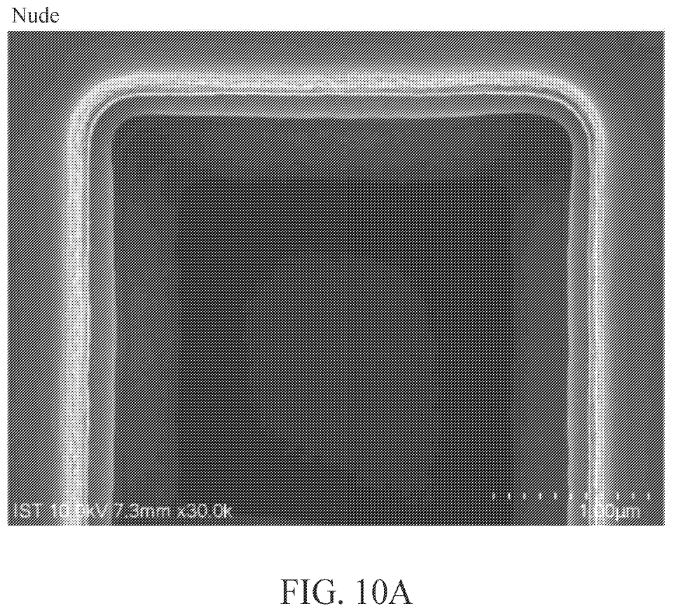

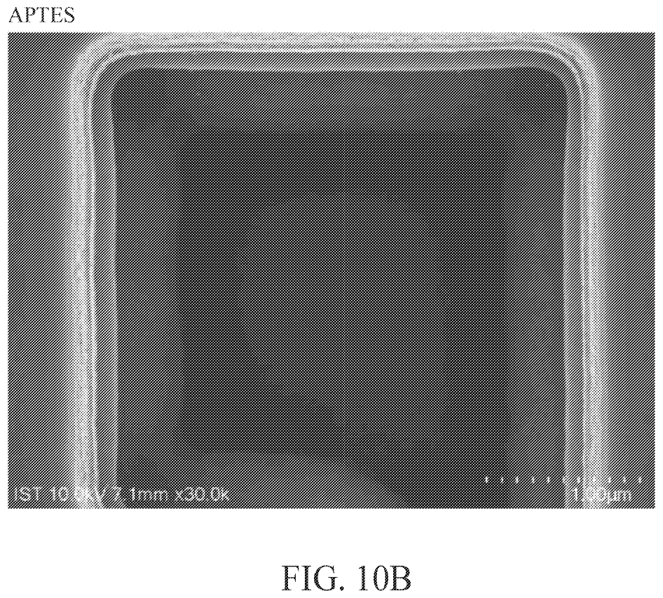

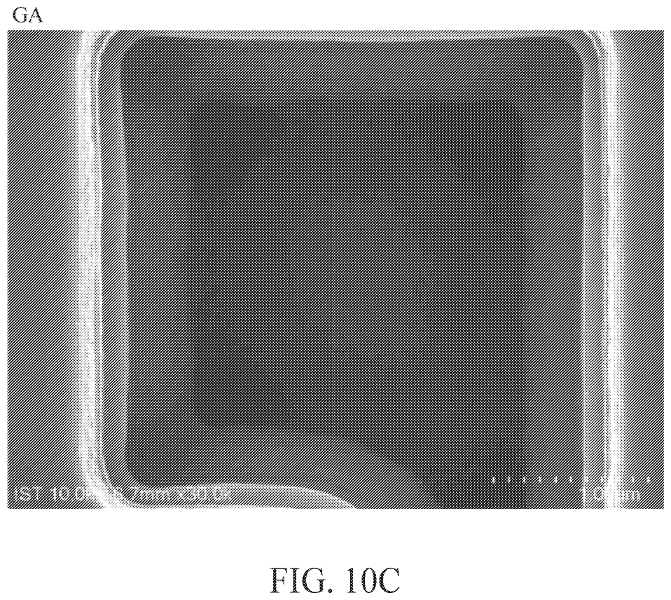

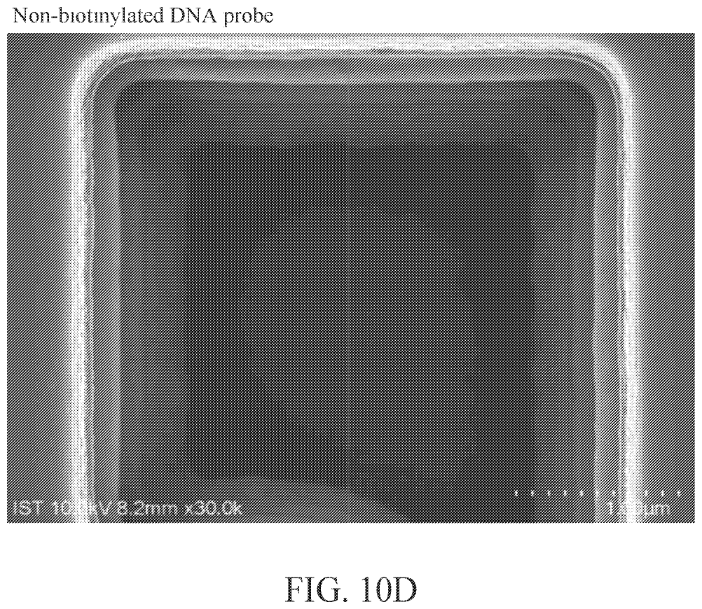

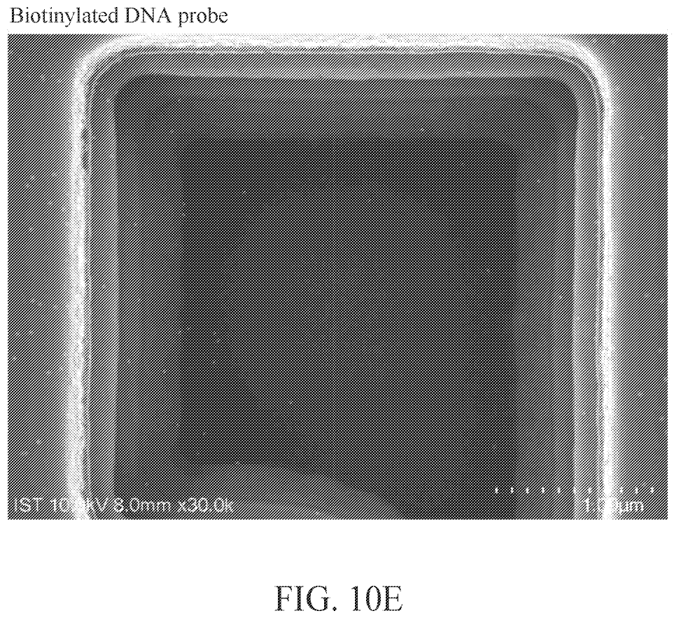

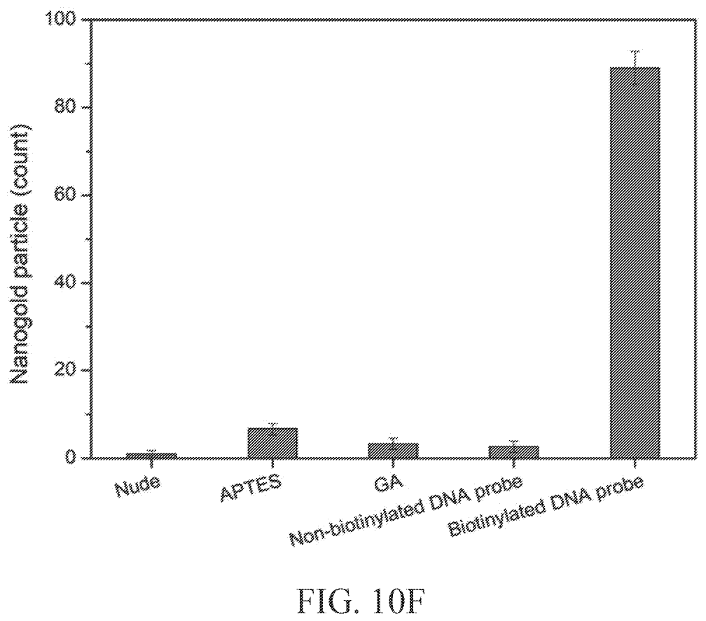

Verification of Surface Modification by SEM: the nanogold particles conjugated on the DNA probe can be observed by SEM. The DNA probe is modified with biotin on the 3′ terminal and is capable of connecting with streptavidin-conjugated nanogold. After the steps of surface modification, the chips are incubated with the streptavidin-conjugated nanogold for 3 hours at the end of each step in surface modification. According to FIG. 10A to FIG. 10C, the SEM images show that there are no nanogold particles observed on the surface of the chip after the chip cleaning, APTES modification, and GA-modification. FIG. 10D shows that no nanogold particles are observed on the surface of the chip after the non-biotinylated DNA probe is immobilized on the chip. As shown in FIG. 10E, after the biotinylated DNA probe immobilized on the chip, many nanogold particles are observed on the surface of the chip. FIG. 10F shows the counts of the nanogold particles by Image J.

The gold nanoparticles are observed by SEM to verify the surface modification and probe immobilization. The streptavidin-conjugated nanogold particles bind with the specific DNA captured probe which is modified with the biotin group on the 3′ terminal. Thus, no nanogold particles are observed on the Nude, APTES-modification, and GA-modification. After the immobilization of the DNA probe, there are many nanogold particles on the surface of the chip.

### Specificity to Different Lengths of DNA

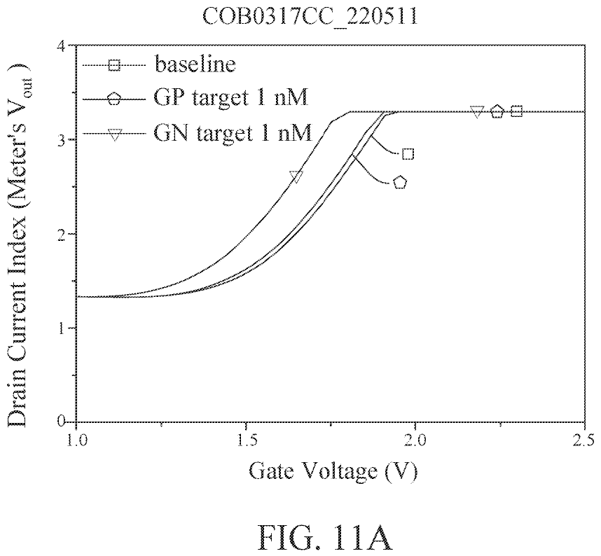

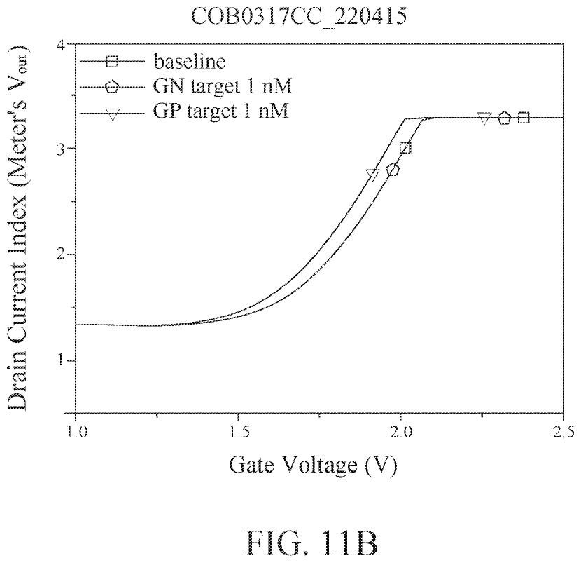

Synthetic DNA Target having 55 base Pairs: the target DNA having 55 base pairs were synthesized by Bio-Protech Technology Enterprise. The target DNA having 55 base pairs is detected by the sensor of the present disclosure, and the result is shown in FIG. 11. In FIG. 11A, the gram-negative probe (GNP) is immobilized on sensing areas of the EGFET, the baseline is first measured in the 10 mM BTP buffer, then the negative control sample 1 nM (gram-positive target DNA having 55 base pairs) is added to the well and incubated for 30 minutes, the I_{D}-V_{G} curve (GP target 1 nM) is close to the baseline. Finally, the target sample 1 nM (gram-negative target DNA having 55 base pairs) is added to the well and incubated for 30 minutes, causing the I_{D}-V_{G} curve to shift to the left (GN target 1 nM). In FIG. 11B, the gram-positive probe (GPP) is immobilized on sensing areas of the EGFET, the baseline is established, then the negative control sample 1 nM (gram-negative target DNA having 55 base pairs) is added to the well and incubated for 30 minutes to obtain the I_{D}-V_{G} curve (GN target 1 nM) which is close to the baseline. Finally, the target sample 1 nM (gram-positive target DNA having 55 base pairs) is added to the well and incubated for 30 minutes, causing the I_{D}-V_{G} curve (GP target 1 nM) to shift to the left.

As shown in FIG. 11A and FIG. 11B, the DNA probe only hybridized with complementary DNA, causing the I_{D}-V_{G} curve to shift. The results show that sensor of the present disclosure demonstrates good specificity to the DNA target having 55 base pairs.

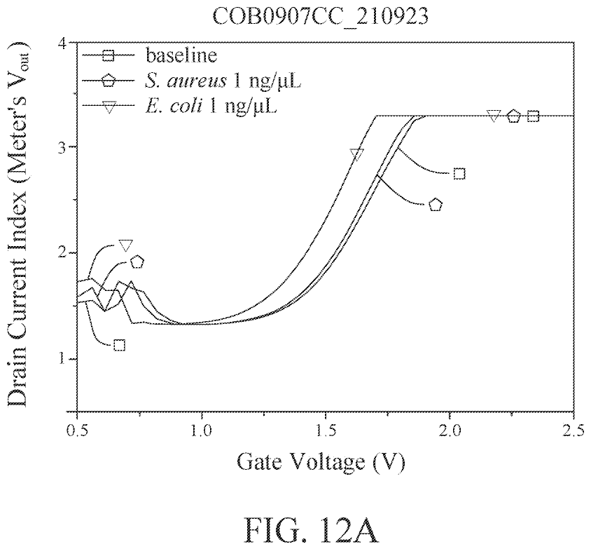

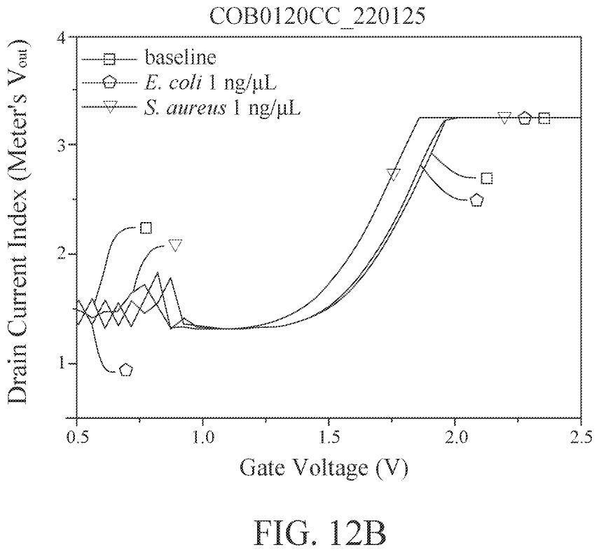

Genomic DNA (gDNA) Target: the gDNA is extracted from E. coli and S. aureus by DNA Mini Kit (Qiagen). The gDNA is heated at 95° C. for 10 minutes and cold shocked for 3 minutes for DNA denaturation and fragmentation. In FIG. 12A, the gram-negative probe (GNP) is immobilized on sensing areas of the EGFET, the baseline is first measured in the 10 mM BTP buffer, then the negative control sample 1 ng/μL (gDNA of S. aureus) is added to the well, the I_{D}-V_{G} curve is consistent with the baseline. Finally, the target sample 1 ng/μL (gDNA of E. coli) is added to the well, causing the I_{D}-V_{G} curve to shift to the left. In FIG. 12B, the gram-positive probe (GPP) is immobilized on sensing areas of the EGFET, the baseline is established, then the negative control sample 1 ng/μL (gDNA of E. coli) is added and the I_{D}-V_{G} curve is close to the baseline. Then, the target sample of 1 ng/μL (gDNA of S. aureus) caused the I_{D}-V_{G} curve to shift to the left.

As shown in FIG. 12, only the target that perfectly matches the DNA probe can induce the I_{D}-V_{G} curve to shift. The results show that using 16S rRNA sequences as a DNA probe on the EGFET can differentiate the gDNA samples.

The sensor of the present disclosure can distinguish between the target DNAs having 55 base pairs at 1 nM, and can distinguish between genomic DNAs at 1 ng/L. As shown in FIG. 11 and FIG. 12, the size of the DNA sequence would not affect the specificity of the sensor of the present disclosure.

### Sensitivity to Different Lengths of DNA

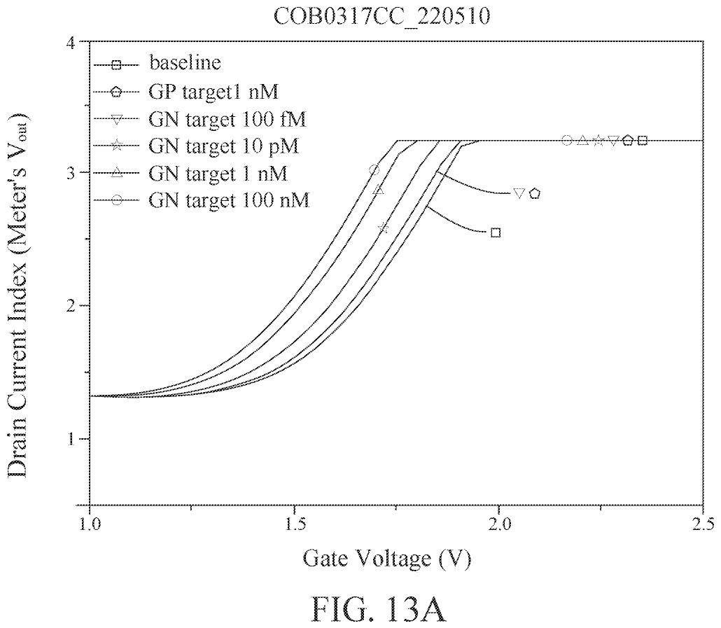

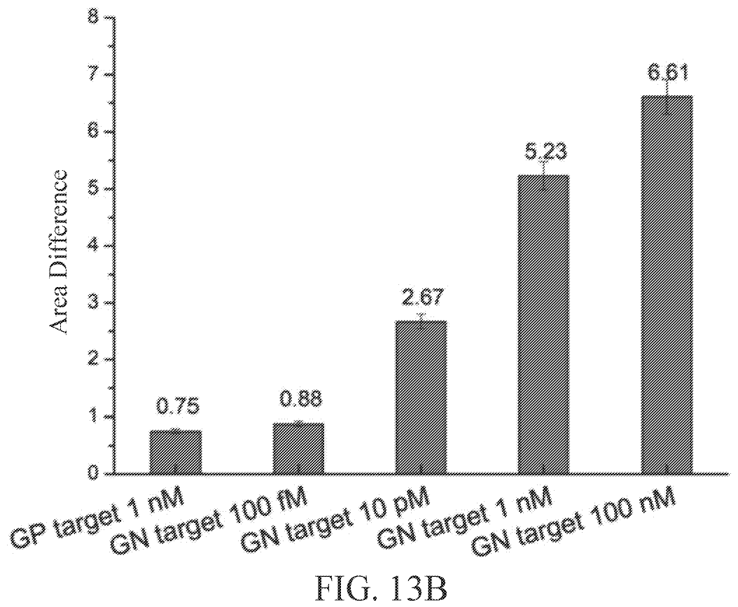

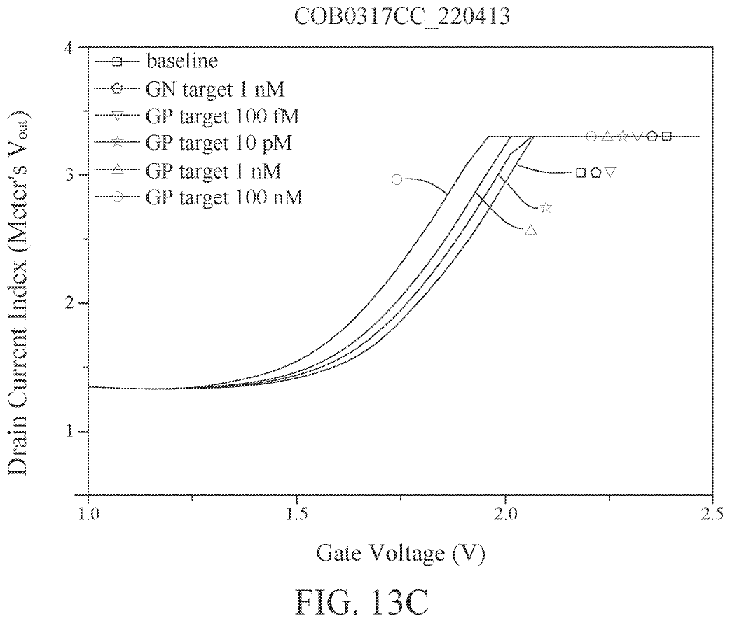

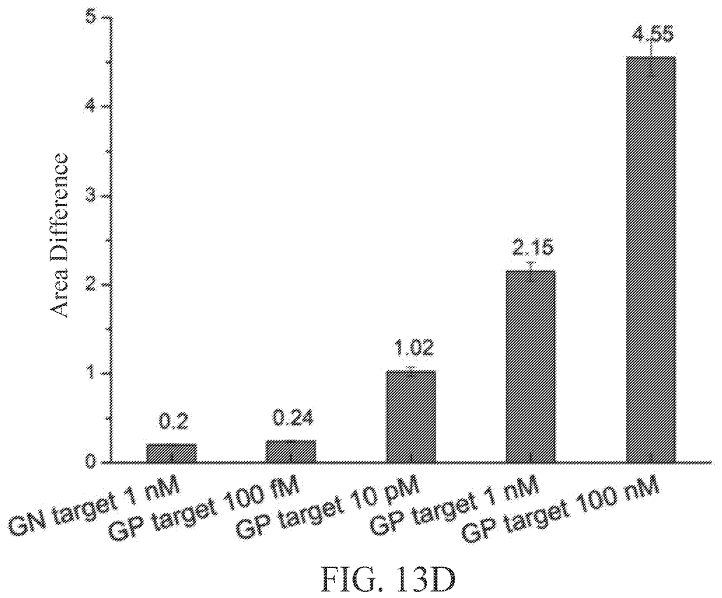

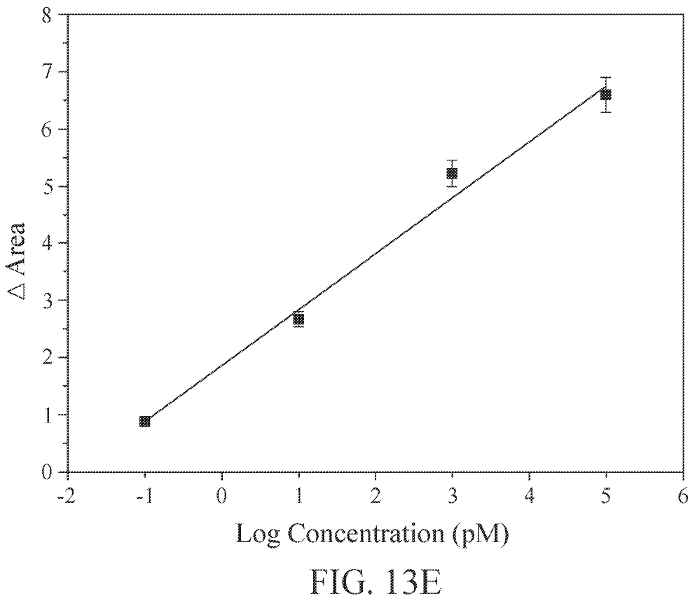

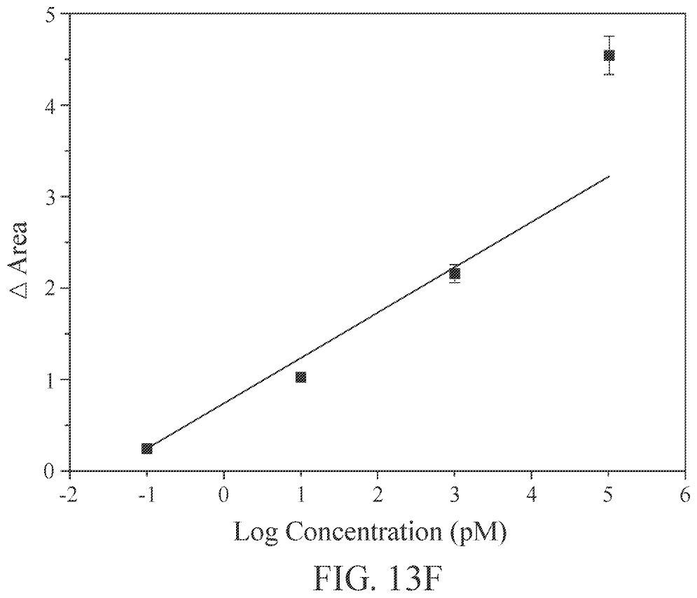

Synthetic Target DNA having 55 base Pairs: the gram-negative probe is used to detect the target DNA having 55 base pairs at 100 fM, 10 pM, 1 nM, and 100 nM on EGFET, and the results are shown in FIG. 13A. The gram-negative probe (GNP) is immobilized on sensing areas of the EGFET, the baseline is measured in the 10 mM BTP buffer, and the I_{D}-V_{G} curve of the negative control 1 nM is close to the baseline. The 100 fM of the target DNA having 55 base pairs didn't induce the same shift as control. The 10 pM of the target DNA having 55 base pairs caused the slightly shifted I_{D}-V_{G} curve. The 1 nM and 100 nM of the target DNA having 55 base pairs induces curve with a greater shift. The area differences are 0.75, 0.88, 2.67, 5.23, and 6.61, respectively, shown in FIG. 13B. The sensitivity slope graph extracted from the hybridization test of the gram-negative target is shown in FIG. 13E. These results show that the short DNA sample requires a concentration of at least 10 pM for detection.

The gram-positive probe is used to detect the target DNA having 55 base pairs at 100 fM, 10 pM, 1 nM, and 100 nM on EGFET, as shown in FIG. 13C. The gram-positive probe (GPP) is immobilized on sensing areas of the EGFET, the baseline is measured in the 10 mM BTP buffer, and the I_{D}-V_{G} curve of the negative control 1 nM is close to the baseline. 100 fM of the target DNA having 55 base pairs didn't induce a shift. 10 pM of the target DNA having 55 base pairs caused the slightly shifted I_{D}-V_{G} curve. 1 nM and 100 nM of the target DNA having 55 base pairs induced I_{D}-V_{G} curves with a greater shift. The area differences are 0.20, 0.24, 1.02, 2.15, and 4.55 respectively are shown in FIG. 13D. The sensitivity slope graph extracted from the hybridization test of the gram-positive target is shown in FIG. 13F.

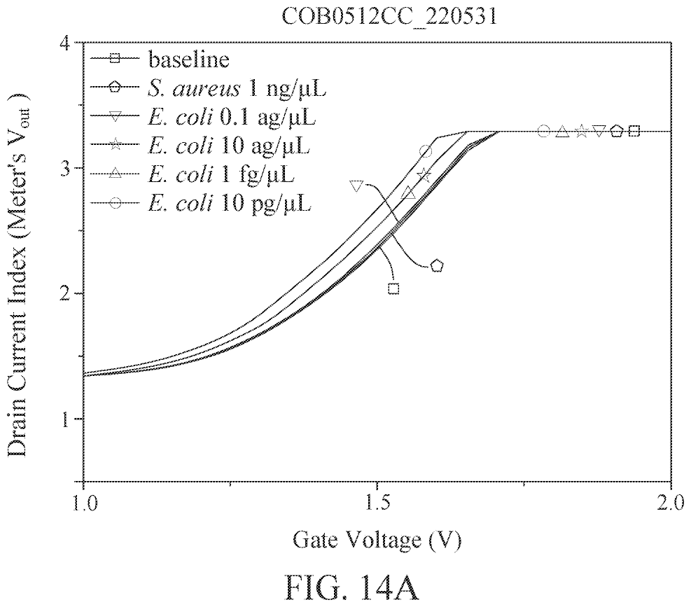

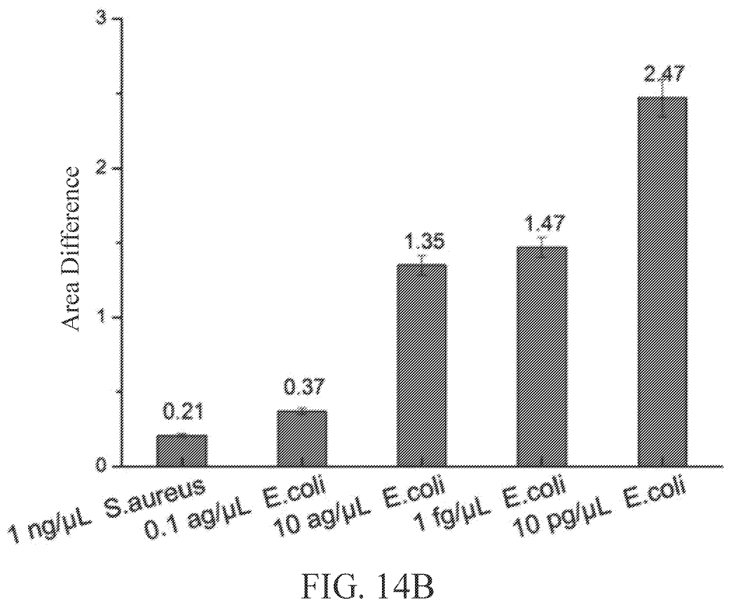

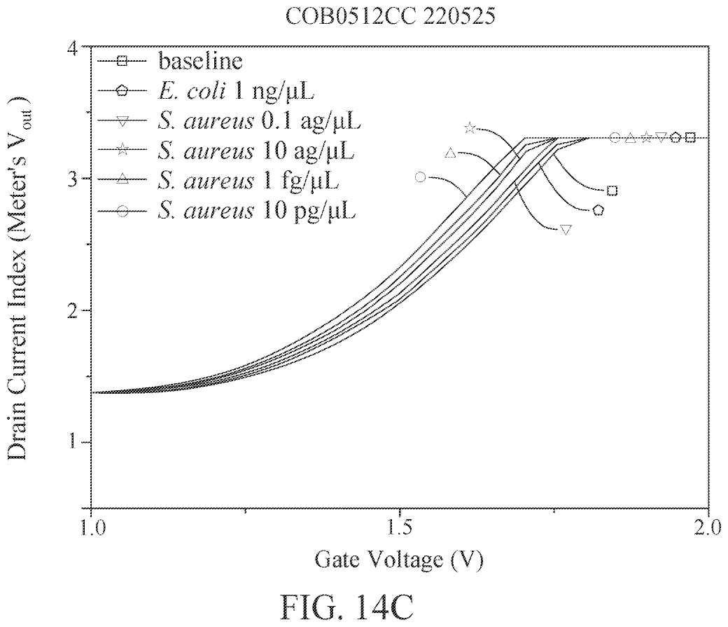

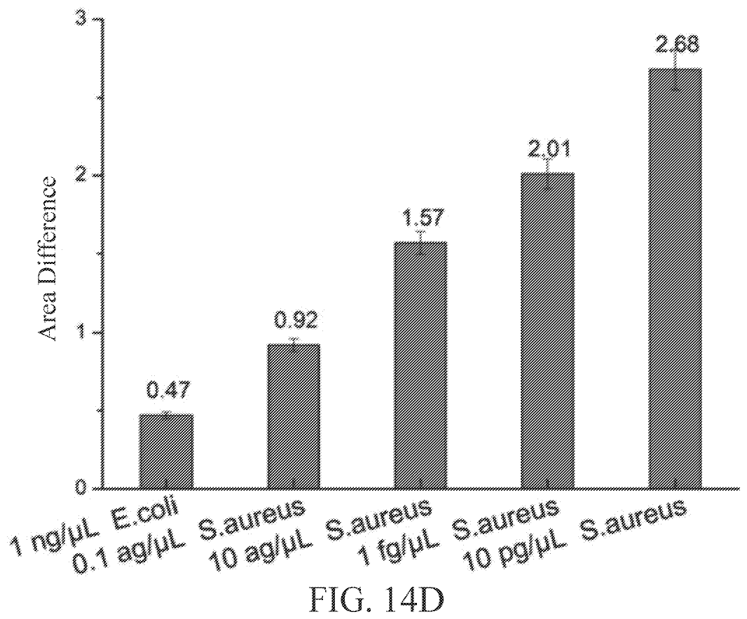

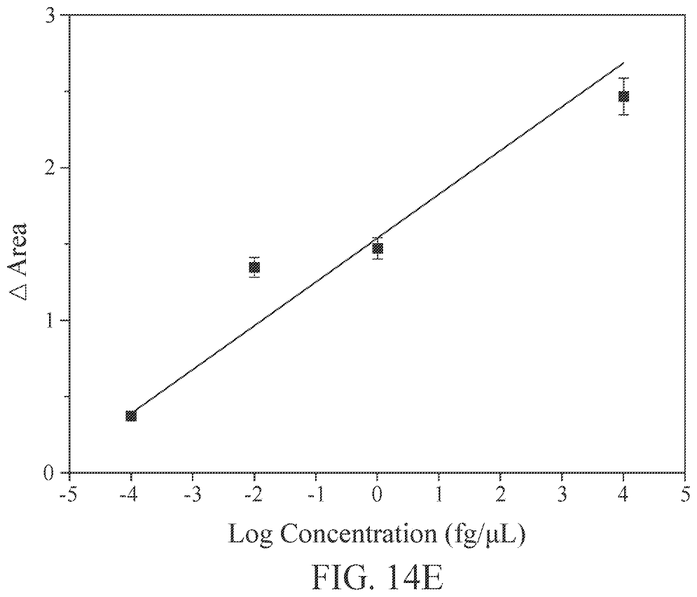

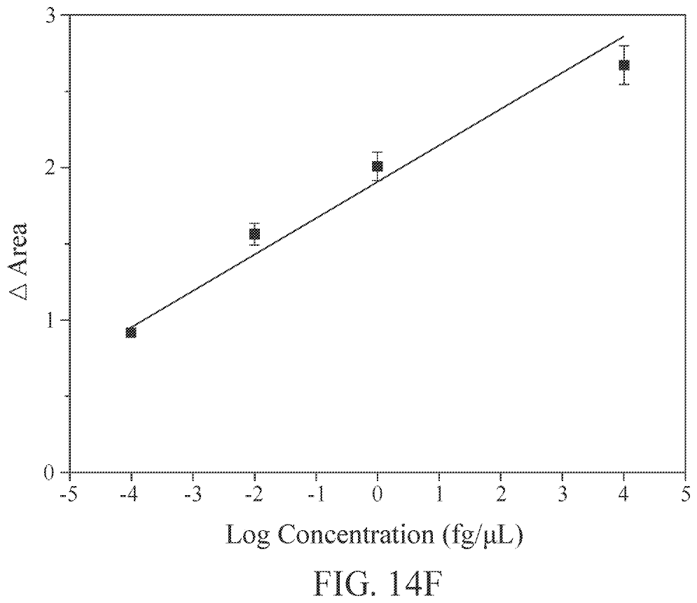

Genomic DNA (gDNA) Target: the gram-negative probe is used to detect the E. coli (gram-negative) gDNA at 0.1 ag/μL, 10 ag/μL, 1 fg/μL, and 10 pg/μL on EGFET are shown in FIG. 14A. The gram-negative probe (GNP) is immobilized on sensing areas of the EGFET, the baseline is measured in the 10 mM BTP buffer, and the I_{D}-V_{G} curve of the negative control 1 ng is close to the baseline. When the gDNA concentration increased from 0.1 ag/μL to 10 pg/μL, the I_{D}-V_{G} curve gradually shifted to the left. The area differences between the different concentrations of gDNA is 0.21, 0.37, 1.35, 1.47, and 2.47 respectively are shown in FIG. 14B. The sensitivity slope graph extracted from the hybridization test of the E. coli gDNA is shown in FIG. 14E. These results show that the E. coli gDNA sample requires a concentration of at least 10 ag/μL for detection.

The gram-positive probe is used to detect the S. aureus (gram-positive) gDNA at 0.1 ag/μL, 10 ag/μL, 1 fg/μL, and 10 pg/μL on EGFET are shown in FIG. 14C. The gram-positive probe (GPP) is immobilized on sensing areas of the EGFET, the baseline is measured in the 10 mM BTP buffer, and the I_{D}-V_{G} curve of the negative control 1 ng is close to the baseline. When the gDNA concentration increased from 0.1 ag/μL to 10 pg/μL, the I_{D}-V_{G} curve gradually shifted to the left. The area differences between the different concentrations of gDNA are 0.47, 0.92, 1.57, 2.01, and 2.68 respectively shown in FIG. 14D. The sensitivity slope graph extracted from the hybridization test of the S. aureus gDNA is shown in FIG. 14F. These results show that the S. aureus gDNA sample requires a concentration of at least 10 ag/μL for detection.

10 pM of the target DNA having 55 base pairs is calculated with the formula (1-2) and converted into a correlation with the DNA concentration measured by Nanodrop. The result shows that the concentration detection limit after conversion by the formula is 0.35 pg/μL. By using the formula (1-2), the detection limit of gDNA is 10 ag/μL.

$$M ( mole / L ) = Amount ( ng / μ L ) / ( base pairs \times 650 ) \qquad ( formula 1 - 2 )$$

Recently, the studies have demonstrated that the efficiency of the sensor platform for target detection depends on the capability of the receptor-target pair to induce a change in the surface charge density on the transducer layer, and there are two approaches to explain the changes in surface potential modulating effect theories (Schöning et al., Label-Free Biosensing. Advanced Materials, Devices and Applications, Springer International Publishing, 2018). A direct effect of modulating the surface-charge density upon binding of charged molecules can be described by the Poisson-Boltzmann theory considering a charged bimolecular layer on top of the sensor surface. In the Poisson-Boltzmann theory, the negative charges of nucleic acid within the Debye length can alter the surface charge density on the surface of the biosensors that results in detection of signal. Another approach is ion distribution theory, which describes an indirect effect in the binding of charged biomolecules that changes the potential of the sensor and thereby modulates the ion concentration near the surface.

In FIG. 13 and FIG. 14, the detection limit of the EGFET for the target DNA having 55 base pairs is 10 pM (0.35 pg/μL). The bacterial gDNA can be detected at 10 ag/μL by EGFET. These results indicate that the EGFET can detect different lengths of DNA sequences. In addition, the long DNA sequences have a better detection limit than the short DNA sequences. Although longer sequences of nucleic acids can be detected at lower concentrations, the shift of the electrical signal is relatively smaller compared to shorter sequences. Because only a few long nucleic acids can bind on the surface of the chip, the lengths of DNA may affect the rate and efficiency of DNA hybridization on the chip (Peplies et al., Applied and environmental microbiology, 2003. 69(3): p. 1397-1407; Peytavi et al., Biotechniques, 2005. 39(1): p. 89-96).

Dilution of Finger-Prick Blood with Different Buffers

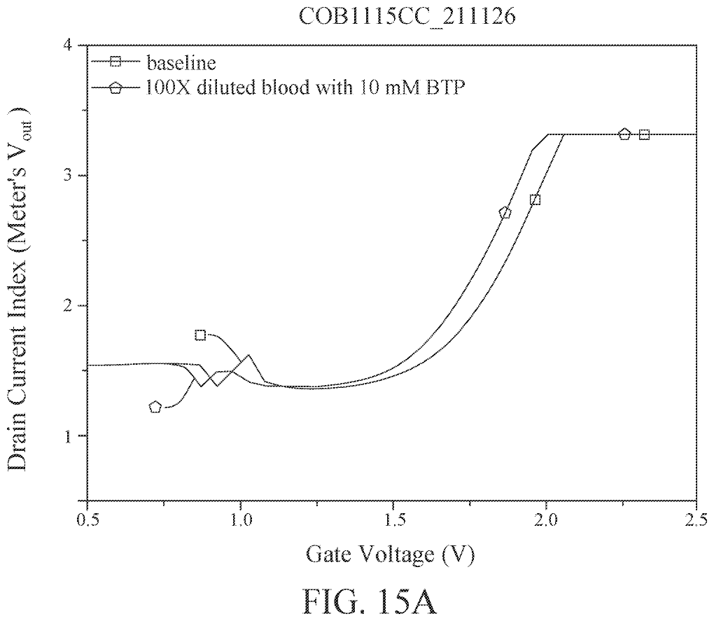

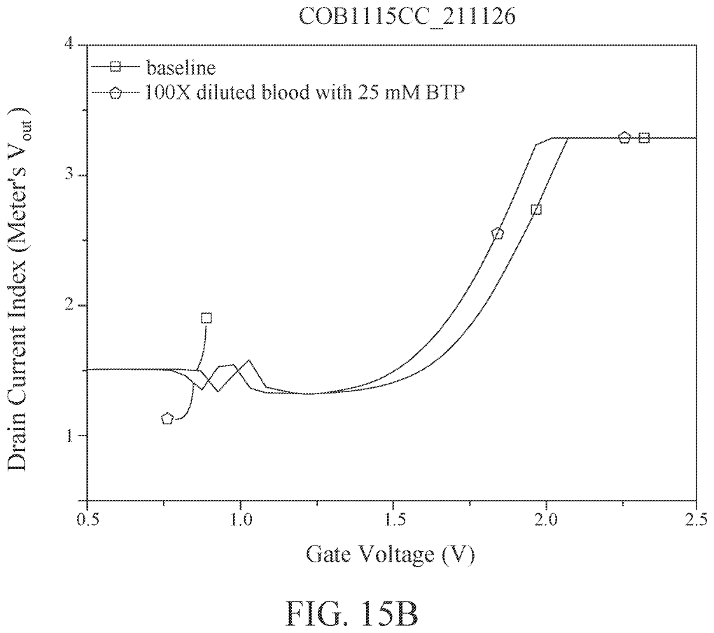

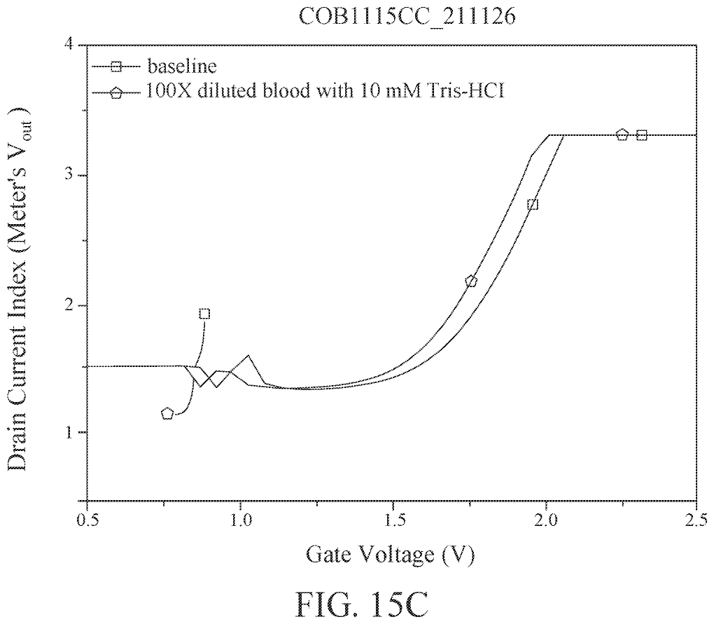

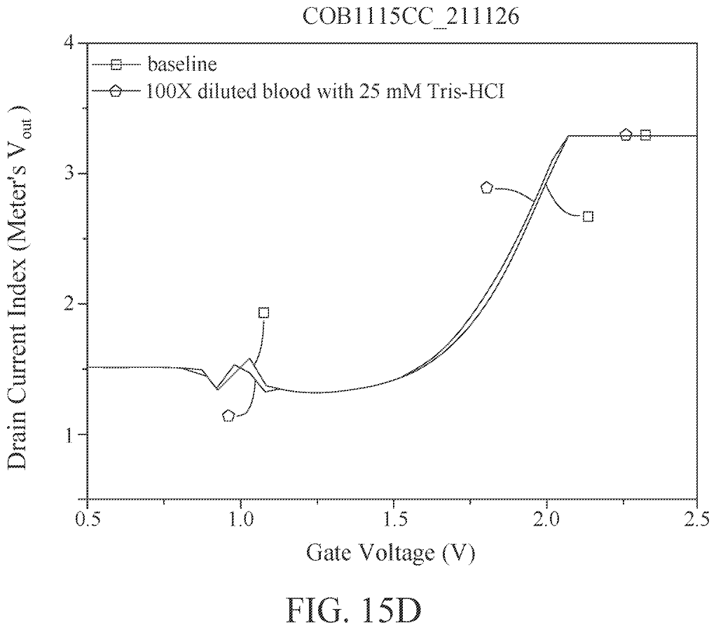

The finger-prick blood was diluted with different buffer solutions before performing nucleic acid testing in whole blood (from the human fingertip blood). The detection was performed to determine which solution buffer has less nonspecific interaction on the nude chip. The blood was diluted with 10 mM BTP buffer, 25 mM BTP buffer, 10 mM Tris-HCl buffer, and 25 mM Tris-HCl, respectively. The results are shown in FIG. 15, after washing with 10 mM BTP buffer and 10 mM BTP+0.05% tween 20, the blood diluted with the 10 mM BTP buffer, 25 mM BTP buffer, and 10 mM Tris-HCl buffer, respectively, can't be washed, and all of them have some non-specific binding on the surface of the EGFET (FIG. 15A to FIG. 15C). In FIG. 15D, the non-specific binding caused by the blood diluted with 25 mM TrisHCl buffer can be washed, and the I_{D}-V_{G} curve shifted to the baseline.

Because no anticoagulant is added is the dilution buffer of the present disclosure, it may cause some nonspecific binding on the surface of the chip after the blood is diluted for detection. The detection results show that when the blood is diluted with 25 mM Tris-HCl buffer, there are less non-specific interactions on the nude chip. Based on these results, the present disclosure provides a method for detecting the gDNA of bacteria or bacteria samples in the diluted blood.

Specificity to Bacteria gDNA Spike in 100× Diluted Finger-Prick Blood

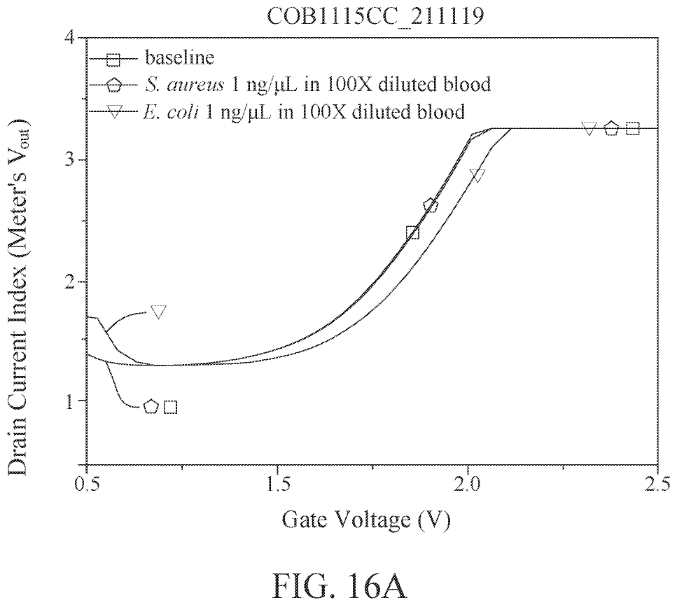

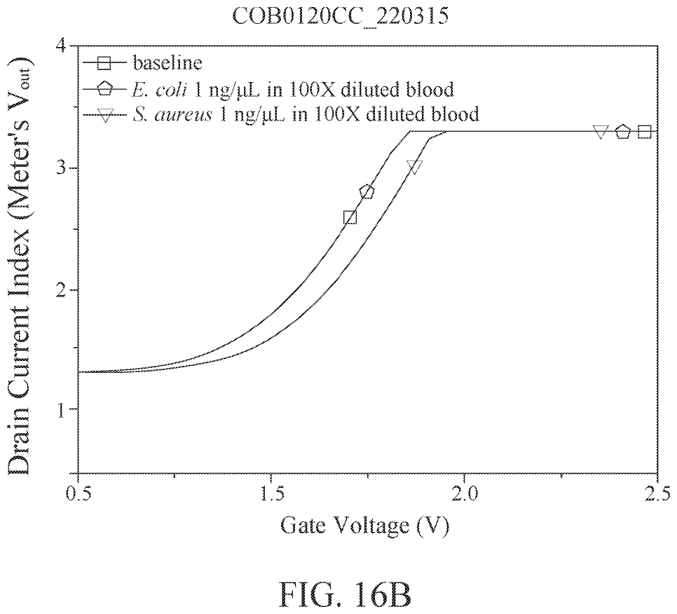

The gDNA is extracted from E. coli and S. aureus by DNA Mini Kit (Qiagen). The gDNA is heated at 95° C. for 10 minutes and cold shocked for 3 minutes for DNA denaturation and fragmentation. The gDNA spike in the 100× diluted finger-prick blood with 25 mM Tris-HCl buffer. In FIG. 16A, the gram-negative probe (GNP) is immobilized on sensing areas of the EGFET, the baseline is measured in the 10 mM BTP buffer, and then a drop of 100× diluted finger-prick blood is loaded into the well to establish a new baseline. The negative control sample 1 ng/μL (gDNA of S. aureus) is added to the well, and the I_{D}-V_{G} curve is consistent with the new baseline. Subsequently, the target sample 1 ng/μL (gDNA of E. coli) is added to the well, causing the I_{D}-V_{G} curve to shift to the right. In FIG. 16B, the gram-positive probe (GPP) is immobilized on sensing areas of the EGFET, the baseline is established, and the 100× diluted finger-prick blood is loaded into the well to establish a new baseline. The negative control sample 1 ng/μL (gDNA of E. coli) is added, and the I_{D}-V_{G} curve is close to the new baseline. Subsequently, the target sample 1 ng/μL (gDNA of S. aureus) caused the I_{D}-V_{G} curve to shift to the right.

In FIG. 16, the results show that using 16S rRNA sequences as a DNA probe on the EGFET can differentiate the gDNA samples in blood, and diluting the blood with 25 mM Tris-HCl buffer results in few or no non-specific binding on the surface of the chip.

Sensitivity to Bacteria gDNA Spike-In 100× Diluted Finger-Prick Blood

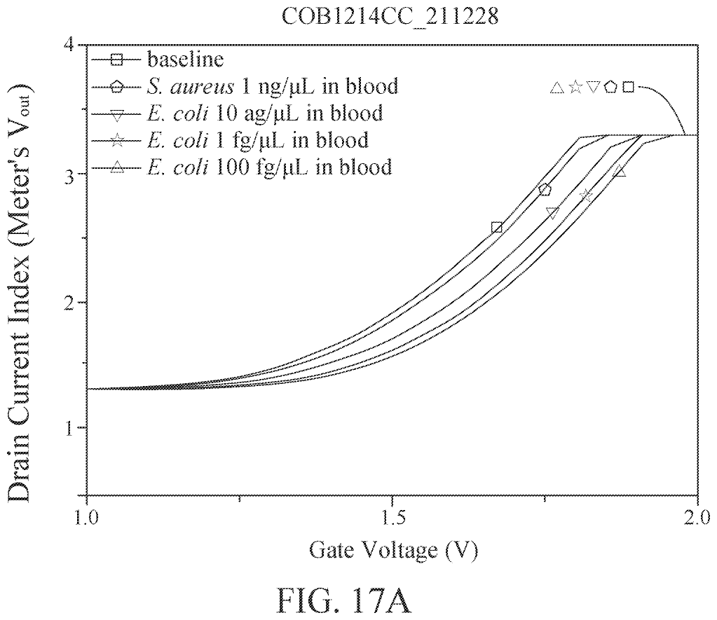

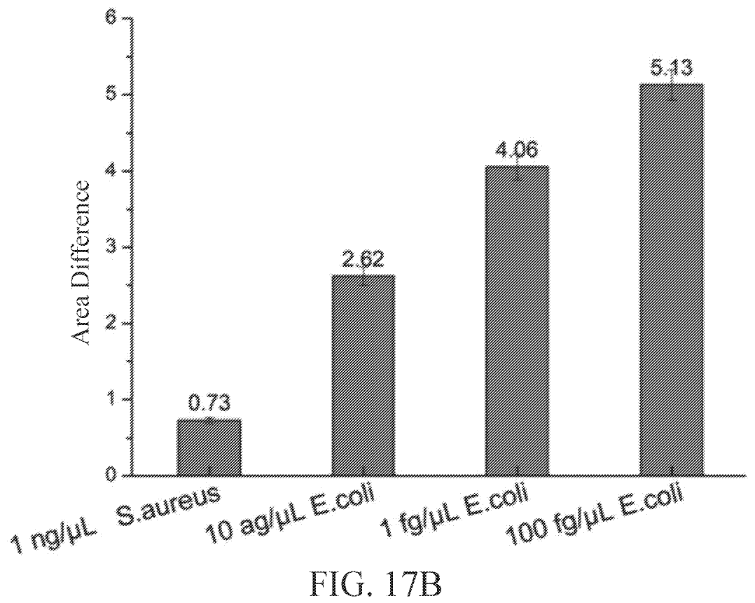

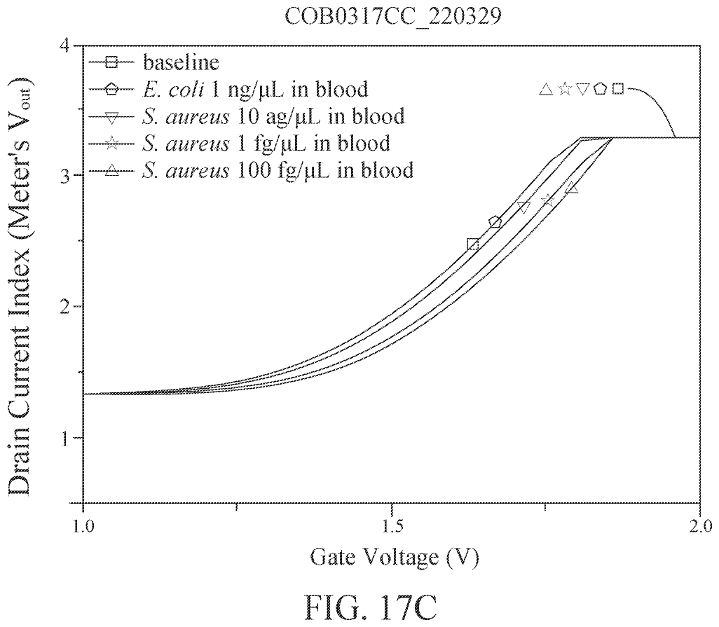

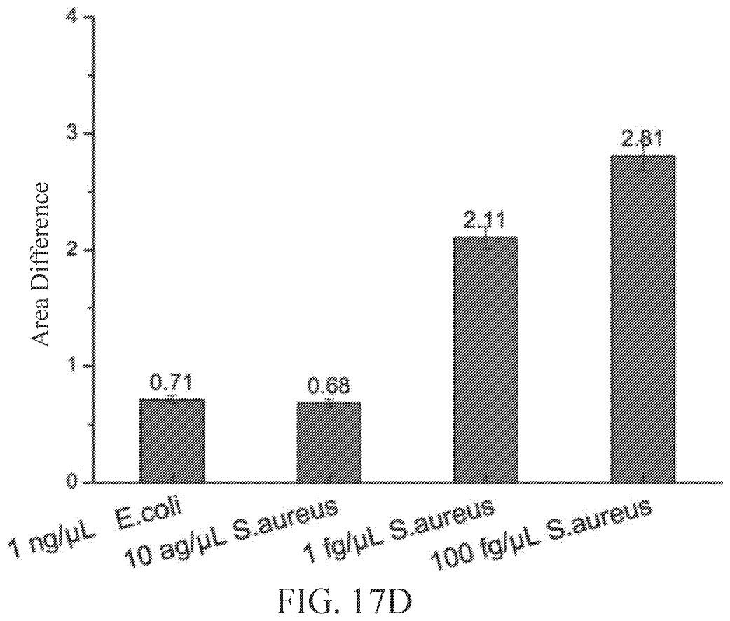

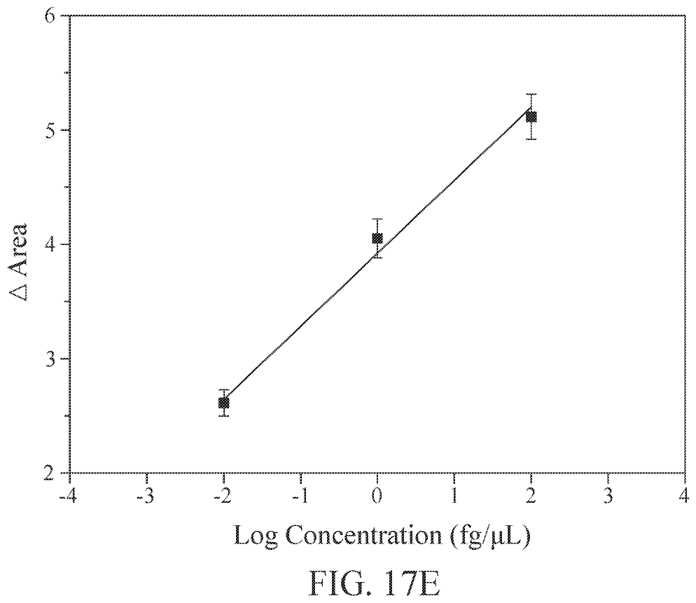

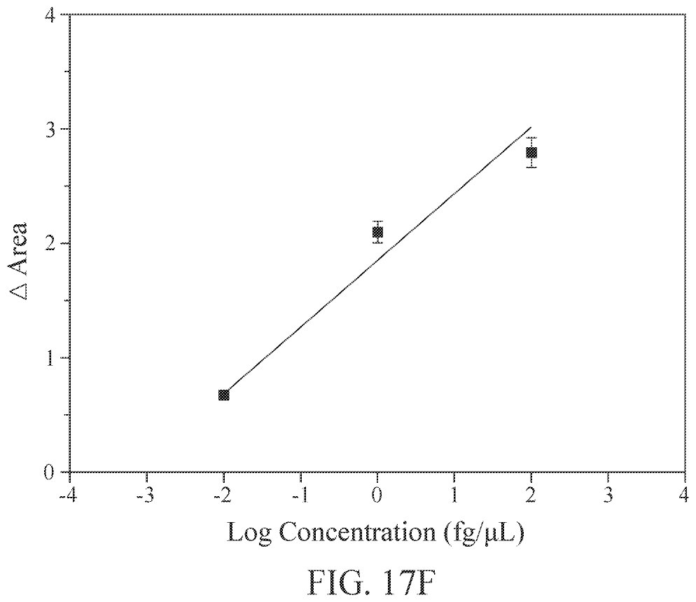

The gram-negative probe was used for detecting the E. coli (gram-negative) gDNA at 10 ag/μL, 1 fg/μL, and 100 fg/μL in whole blood on EGFET. The results are shown in FIG. 17A. The gram-negative probe (GNP) was immobilized on sensing areas of the EGFET. When the baseline was stable, the 100× diluted finger-prick blood was added into the well to establish a new baseline, and the I_{D}-V_{G} curve of the negative control 1 ng/μL was close to the baseline. When the target gDNA concentration was increased from 10 ag/μL to 100 fg/μL, the I_{D}-V_{G} curve gradually shifted to the right. The area differences between the different concentrations of gDNA are 0.73, 2.62, 4.06, and 5.13 respectively, as shown in FIG. 17B. The sensitivity slope graph extracted from the hybridization test of the E. coli gDNA in the blood is shown in FIG. 17E. These results show that the E. coli gDNA sample requires a concentration of at least 10 ag/μL for detection.

The gram-positive probe was used for detecting the S. aureus (gram-positive) gDNA at 10 ag/μL, 1 fg/μL, and 100 fg/μL in 100× diluted finger-prick blood on EGFET. The results are shown in FIG. 17C. The gram-positive probe (GPP) is immobilized on sensing areas of the EGFET. When the baseline is stable, the 100× diluted finger-prick blood is added into the well to establish a new baseline, and the I_{D}-V_{G} curve of the negative control 1 ng/μL is close to the baseline. When the target gDNA concentration was increased from 10 ag/μL to 100 fg/μL, the I_{D}-V_{G} curve gradually shifted to the right. The comparison of area differences between the different concentrations of gDNA is 0.71, 0.68, 2.11, and 2.81 respectively as shown in FIG. 17D. The sensitivity slope graph extracted from the hybridization test of the S. aureus gDNA in the blood is shown in FIG. 17F. These results show that the S. aureus gDNA sample requires a concentration of at least 1 fg/μL for detection.

The detection is carried out by gDNA spiked-in 100× diluted finger-prick blood to simulate the detection performed on samples of patients with sepsis. It is confirmed that the bacterial gDNA in the blood can be detected by the sensor of the present disclosure. As shown in FIG. 17, the detection limit for gDNA spiked-in 100× diluted finger-prick blood is 1 fg/μL and 10 ag/μL, respectively, by using the gram-positive probe and gram-negative probe.

### Sensitivity to Bacteria Spike-In 100× Diluted Finger-Prick Blood

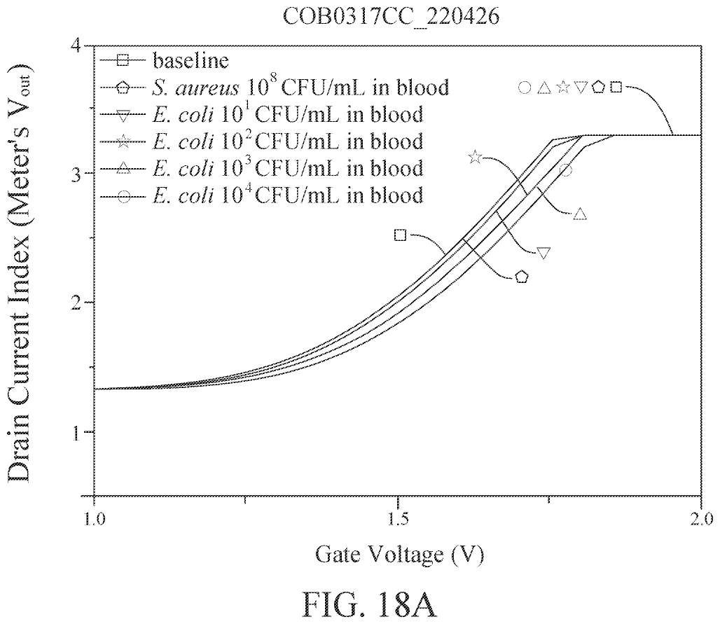

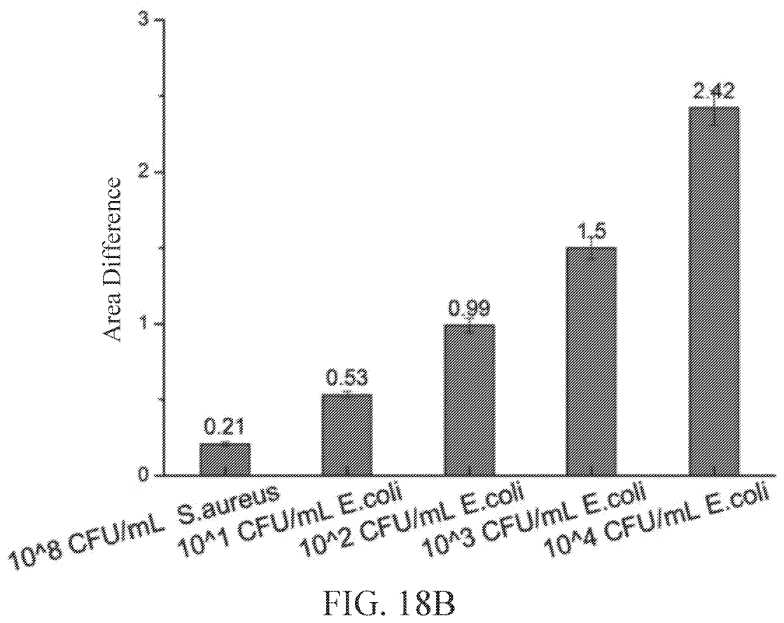

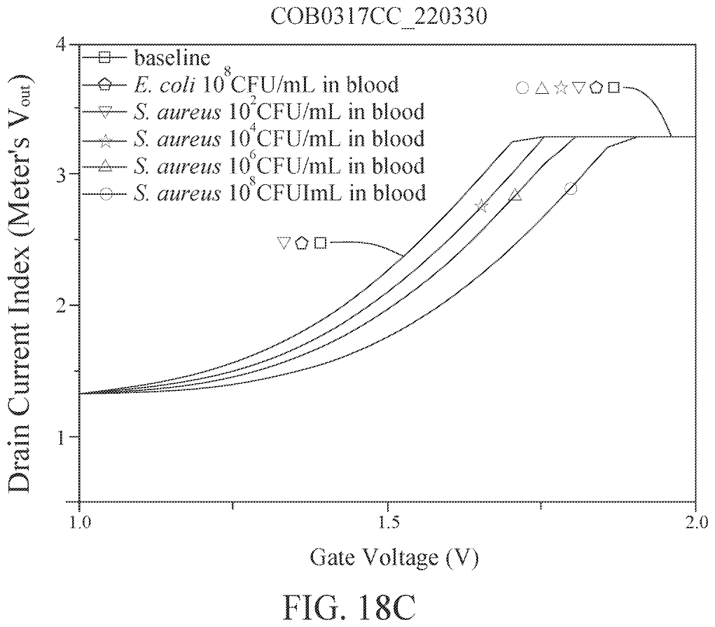

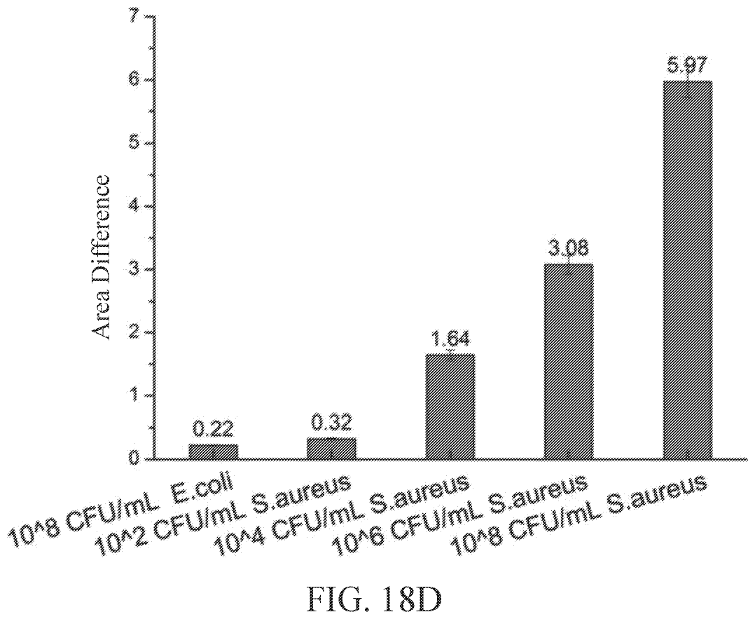

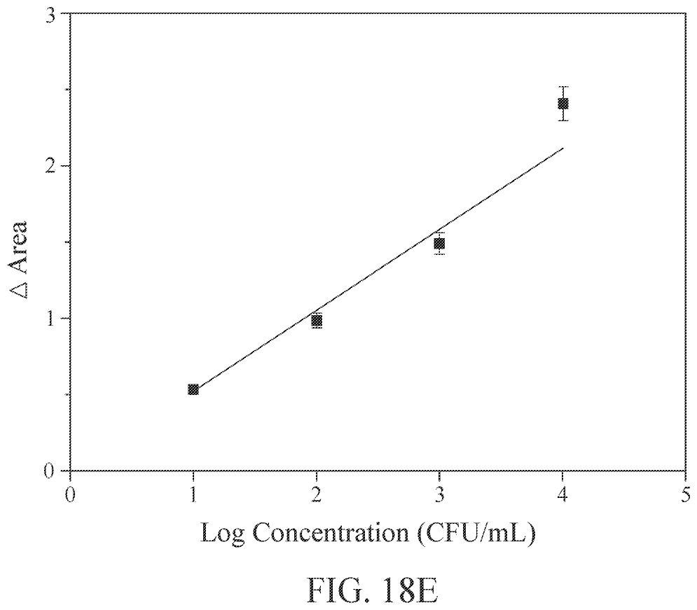

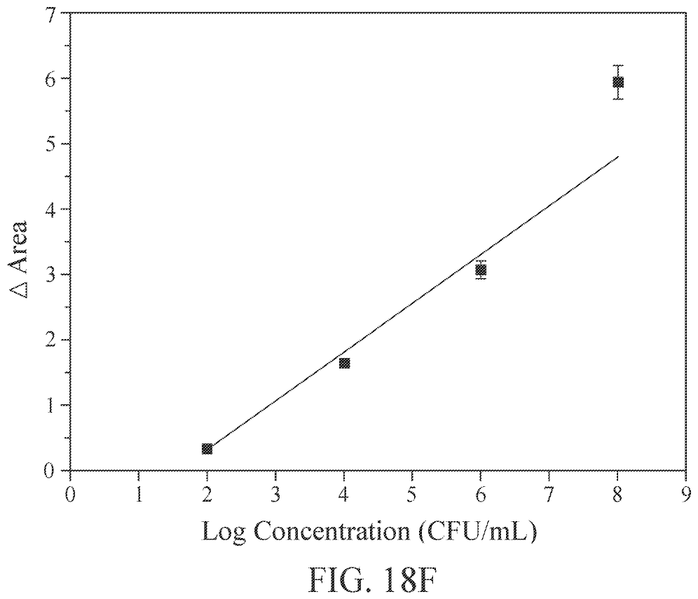

After bacterial quantification, all samples are diluted to various cell densities in the 100× diluted whole blood with 25 mM Tris-HCl buffer. After that, these samples are heated lysis at 95° C. for 10 minutes and centrifuged and aspirated with the supernatant for detection. In FIG. 18A, the gram-negative probe (GNP) is immobilized on sensing areas of the EGFET, when the baseline is stable, the 100× diluted finger-prick blood is added into the well to establish a new baseline, and the I_{D}-V_{G} curve of the negative control S. aureus 10⁸ CFU/mL is close to the baseline. When the E. coli gDNA concentration was increased from 10 CFU/mL to 10⁴ CFU/mL, the I_{D}-V_{G} curve gradually shifted to the right. The comparison of area differences between the different concentrations of cell density is 0.21, 0.53, 0.99, 1.50, and 2.42 respectively are shown in FIG. 18B. The sensitivity slope graph extracted from the hybridization test of pre-treatment the E. coli sample in the blood is shown in FIG. 18E. These results show that the E. coli samples requires a concentration of at least 10² CFU/mL for detection.

In FIG. 18C, the gram-positive probe (GPP) is immobilized on sensing areas of the EGFET, when the baseline is stable, the 100× diluted finger-prick blood is added into the well to establish a new baseline, and the I_{D}-V_{G} curve of the negative control E. coli 10⁸ CFU/mL is close to the baseline. When the S. aureus gDNA concentration was increased from 102 CFU/mL to 108 CFU/mL, the I_{D}-V_{G} curve gradually shifted to the right. The comparison of area differences between the different concentrations of cell density is 0.22, 0.32, 1.64, 3.08, and 5.97 respectively are shown in FIG. 18D. The sensitivity slope graph extracted from the hybridization test of pre-treatment the S. aureus sample in the blood is shown in FIG. 18F. These results show that the S. aureus samples requires a concentration of at least 10⁴ CFU/mL for detection.

The present disclosure provides another method for detecting bacterial gDNA by pre-treating the specimens from patients with sepsis using heat lysis. As shown in FIG. 18, the detection limit for bacteria samples spiked in 100× diluted finger-prick blood is 10⁴ CFU/mL and 10² CFU/mL, respectively, by using the gram-positive probe and gram-negative probe. The sensor of the present disclosure can differentiate whether the gDNA is low concentration or absent in the blood sample of patients. Accordingly, the present disclosure provides a method and a sensor for detecting sepsis. When a person with sepsis is suffering from an infection, the blood contains disease-causing bacteria. The bacterial DNA in the blood can be extracted for detection. The method and sensor of the present disclosure provide an efficient and fast way to identify the gram properties of bacteria species in the blood of the patient with sepsis.

### Confirmation of the Target DNA by SEM

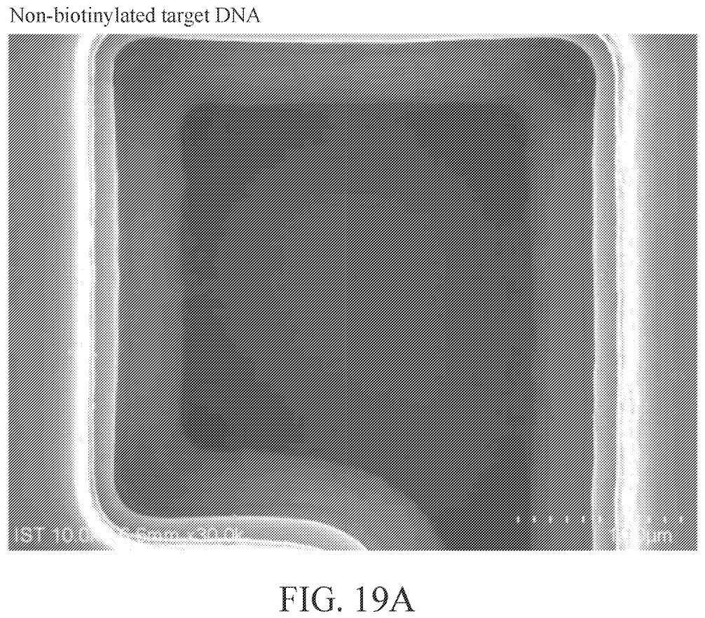

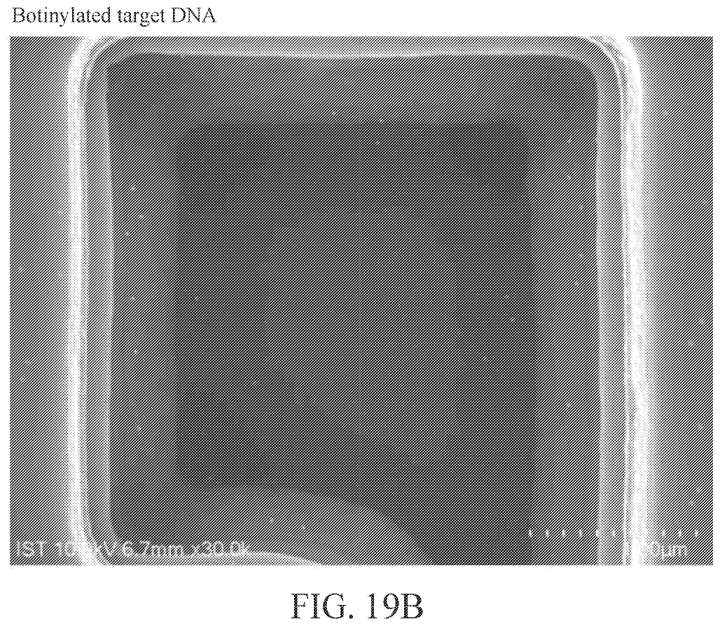

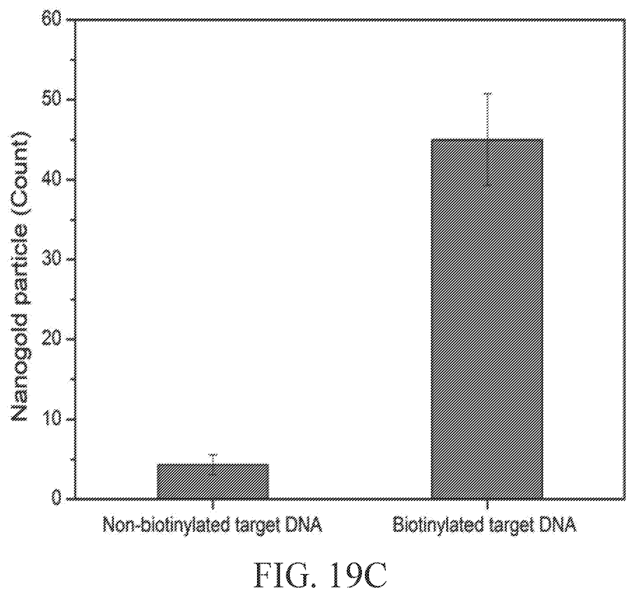

The target DNA was modified with the biotin group on the 3′ terminal and is capable of connecting with streptavidin-conjugated nanogold. FIG. 19A shows that no nanogold particles are observed on the surface of the chip, after incubating the non-biotinylated target DNA for 30 minutes. FIG. 19B shows that a lot of nanogold particles are observed on the surface of the chip, after incubating the biotinylated target DNA for 30 minutes. FIG. 19C shows that the counts of the nanogold particle count by Image J.

In the present disclosure, EGFET of the sensor can detect the different sizes of DNA with sufficient sensitivity. The sensitivity to 55 base pairs and gDNA was 10 pM (0.35 pg/μL) and 10 ag/μL with GNP and GPP, respectively. In the present disclosure, the probes are designed to distinguish gram-bacteria, and two methods are provided to detect specimens from patients with sepsis. In the method of the present disclosure, spiked-in gDNA in 100× diluted blood can be detected wherein sensitivity is 10 ag/μL for E. coli and 1 fg/μL for S. aureus using GNP and GPP, and spiked-in bacteria sample in 100× diluted blood can be detected wherein the sensitivity is 10² CFU/mL for E. coli and 10⁴ CFU/mL for S. aureus using GNP and GPP.

The present disclosure shortens the detection time of sepsis. Rapid identification of pathogens in the blood will facilitate appropriate treatment, reduce mortality, limit the use of broadspectrum antibiotics, and reduce the emergence of drug-resistant bacteria. Molecular detection methods are advantageous because they are not dependent on bacterial growth and have higher sensitivity to detect low concentrations of pathogenic bacteria.

While the present disclosure has been described by means of specific embodiments, numerous modifications and variations could be made thereto by those skilled in the art without departing from the scope and spirit of the present disclosure set forth in the claims.

What is claimed is:

1. A method for detecting bacterial nucleic acid, comprising steps of:
providing an extended gate field-effect transistor (EGFET) having a sensing area;
immobilizing a nucleic acid probe on the sensing area, wherein the nucleic acid probe is specific to Gram-negative bacteria or Gram-positive bacteria;
providing a control sample on the sensing area of the EGFET;
detecting a first electrical signal of the EGFET after the control sample is provided on the sensing area of the EGFET;
providing a sample on the sensing area of the EGFET;
detecting a second electrical signal of the EGFET after the sample is provided on the sensing area of the EGFET; and
comparing the first electrical signal and the second electrical signal to determine whether there is the bacterial nucleic acid in the sample.

2. The method according to claim 1, wherein the nucleic acid probe includes a sequence of SEQ ID NO: 1 which is specific to the Gram-negative bacteria.

3. The method according to claim 1, wherein the Gram-negative bacteria is E. coli.

4. The method according to claim 1, wherein the nucleic acid probe includes a sequence of SEQ ID NO: 2 which is specific to the Gram-positive bacteria.

5. The method according to claim 1, wherein the Gram-positive bacteria is S. aureus.

6. The method according to claim 1, wherein the sample is diluted with Tris-HCl buffer.

7. The method according to claim 6, wherein the Tris-HCl buffer has a concentration of 25 mM.

8. The method according to claim 1, wherein the first electrical signal is converted to a first I_{D}-VG curve, and the second electrical signal is converted to a second I_{D}-V_{G} curve.

9. The method according to claim 8, wherein there is the bacterial nucleic acid in the sample when there is a shift from the first I_{D}-V_{G} curve to the second I_{D}-V_{G} curve.

10. The method according to claim 9, wherein the nucleic acid probe includes a sequence of SEQ ID NO: 1 which is specific to the Gram-negative bacteria, and the bacterial nucleic acid of the Gram-negative bacteria in the sample has a concentration of at least 10 ag/μL.

11. The method according to claim 9, wherein the nucleic acid probe includes a sequence of SEQ ID NO: 2, and the Gram-positive bacteria in the sample has a concentration of at least 1 fg/μL.

12. A method for detecting bacterial nucleic acid, comprising steps of:
providing an extended gate field-effect transistor (EGFET) having a sensing area;
immobilizing a nucleic acid probe on the sensing area, wherein the nucleic acid probe is specific to Gram-negative bacteria or Gram-positive bacteria;
providing a control sample on the sensing area of the EGFET;
detecting a first electrical signal of the EGFET after the control sample is provided on the sensing area of the EGFET;
providing a sample on the sensing area of the EGFET;
detecting a second electrical signal of the EGFET after the sample is provided on the sensing area of the EGFET; and
based on the first electrical signal and the second electrical signal, determining an amount of the bacterial nucleic acid in the sample.

13. The method according to claim 12, wherein the nucleic acid probe includes a sequence of SEQ ID NO: 1 which is specific to the Gram-negative bacteria.

14. The method according to claim 12, wherein the Gram-negative bacteria is E. coli.

15. The method according to claim 12, wherein the nucleic acid probe includes a sequence of SEQ ID NO: 2 which is specific to the Gram-positive bacteria.

16. The method according to claim 12, wherein the Gram-positive bacteria is S. aureus.

17. The method according to claim 12, wherein the sample is diluted with Tris-HCl buffer.

18. The method according to claim 17, wherein the Tris-HCl buffer has a concentration of 25 mM.

19. The method according to claim 12, wherein the first electrical signal is converted to a first I_{D}-V_{G} curve, and the second electrical signal is converted to a second I_{D}-V_{G} curve.

20. The method according to claim 19, further comprising a step of comparing areas of the first I_{D}-V_{G} curve and the second I_{D}-V_{G} curve to determine the amount of the bacterial nucleic acid.

21. The method according to claim 20, wherein the nucleic acid probe includes a sequence of SEQ ID NO: 1 which is specific to the Gram-negative bacteria, and the bacterial nucleic acid of the Gram-negative bacteria in the sample has a concentration of at least 10 ag/μL.

22. The method according to claim 20, wherein the nucleic acid probe includes a sequence of SEQ ID NO: 2, and the Gram-positive bacteria in the sample has a concentration of at least 1 fg/μL.

23. A sensor for detecting bacterial nucleic acid, comprising:
an extended gate field-effect transistor (EGFET) having a sensing area; and
a nucleic acid probe immobilized on the sensing area, wherein the nucleic acid probe is specific to Gram-negative bacteria or Gram-positive bacteria.

24. The sensor according to claim 23, wherein the nucleic acid probe includes a sequence of SEQ ID NO: 1 which is specific to the Gram-negative bacteria.

25. The sensor according to claim 23, wherein the Gram-negative bacteria is E. coli.

26. The sensor according to claim 23, wherein the nucleic acid probe includes a sequence of SEQ ID NO: 2 which is specific to the Gram-positive bacteria.

27. The sensor according to claim 23, wherein the Gram-positive bacteria is S. aureus.

28. The sensor according to claim 23, wherein the bacterial nucleic acid is detected by steps of providing a control sample on the sensing area of the EGFET; detecting a first electrical signal of the EGFET after the control sample is provided on the sensing area of the EGFET; providing a sample on the sensing area of the EGFET; and detecting a second electrical signal of the EGFET after the sample is provided on the sensing area of the EGFET.

29. The sensor according to claim 28, wherein the first electrical signal is converted to a first I_{D}-V_{G} curve, and the second electrical signal is converted to a second I_{D}-V_{G} curve.

30. The sensor according to claim 28, wherein there is the bacterial nucleic acid in the sample when there is a shift from the first I_{D}-V_{G} curve to the second I_{D}-V_{G} curve.

31. The sensor according to claim 28, wherein areas of the first I_{D}-V_{G} curve and the second I_{D}-V_{G} curve are compared to determine an amount of the bacterial nucleic acid.

32. A method for verifying an electrical property of an EGFET, comprising steps of:
providing an EGFET in a first buffer solution with a first pH value;
measuring a first electric signal of the EGFET in the first buffer solution;
providing a second buffer solution with different pH values;
providing the EGFET in the second buffer solution;
measuring a second electrical signal of the EGFET in the second buffer solution; and
converting the first electric signal to a first I_{D}-V_{G} curve and converting the second electrical signal to a second I_{D}-V_{G} curve.

33. The method according to claim 32, wherein one of the first buffer solution, the second buffer solution and the third buffer solution is BTP buffer.

34. The method according to claim 33, wherein the BTP buffer has a concentration of 10 mM.

35. The method according to claim 32, wherein the different pH values include 5, 7 and 8.

36. The method according to claim 35, wherein the EGFET is measured in the buffer solution with the different pH values in the order of pH7, pH5, pH7, pH8, and pH7.

37. The method according to claim 32, wherein the EGFET is a nude EGEFT.

38. A method for verifying surface modification of an EGFET, comprising steps of:
providing an EGFET in a buffer solution with a pH value;
measuring a first electric signal of the EGFET in the buffer solution with the pH value;
modifying the EGFET to form a modified EGFET;
providing the modified EGFET in the buffer solution with the pH value;
measuring a second electrical signal of the modified EGFET in the buffer solution with the pH value;
converting the first electric signal to a first I_{D}-V_{G} curve; and
converting the second electrical signal to a second I_{D}-V_{G} curve.

39. The method according to claim 38, wherein the buffer solution is BTP buffer.

40. The method according to claim 39, wherein the BTP buffer has a concentration of 10 mM.

41. The method according to claim 38, wherein the EGFET is a nude EGEFT.


SEQUENCE LISTING (8 sequences)

SEQ ID NO: 1 - DNA, 37 bp, synthetic construct
  source 1..37: mol_type = other DNA
accgcgaaaagcaccggctacgtaggcggttaagtcg

SEQ ID NO: 2 - DNA, 37 bp, synthetic construct
  source 1..37: mol_type = other DNA
aaggggataactgggaaacgggctaataccgatagat

SEQ ID NO: 3 - DNA, 55 bp, synthetic construct
  source 1..55: mol_type = other DNA
ttgacccgggatttcacacgacttaaccgcctacgtagccggtgcttttcgcggt

SEQ ID NO: 4 - DNA, 55 bp, synthetic construct
  source 1..55: mol_type = other DNA
cctttcttgacatgccaaatctatcggtattagcccgtttcccagttatcccctt

SEQ ID NO: 5 - DNA, 20 bp, synthetic construct
  source 1..20: mol_type = other DNA
accgcgaaaagcaccggcta

SEQ ID NO: 6 - DNA, 20 bp, synthetic construct
  source 1..20: mol_type = other DNA
aggggtgcgaaagcgcggat

SEQ ID NO: 7 - DNA, 20 bp, synthetic construct
  source 1..20: mol_type = other DNA
aaggggataactgggaaacg

SEQ ID NO: 8 - DNA, 20 bp, synthetic construct
  source 1..20: mol_type = other DNA
aggctttggaagtaggagtg